(19) United States
(12) Patent Application Publication
(10) Pub. No.: US 2025/0182892 A1
(43) Pub. Date: Jun. 5, 2025

(54) METHOD OF MONITORING CANCER USING FRAGMENTATION PROFILES

(71) Applicant: Delfi Diagnostics, Inc., Boston, MA (US)

(72) Inventors: Keith Lumbard, Baltimore, MD (US); Laurel Keefer, Baltimore, MD (US); Jacob Carey, Baltimore, MD (US); Alessandro Leal, Baltimore, MD (US); Nicholas C. Dracopoli, Baltimore, MD (US); Lorenzo Rinaldi, Baltimore, MD (US)

(21) Appl. No.: 18/844,348

(22) PCT Filed: Mar. 17, 2023
(86) PCT No.: PCT/US2023/015559

§ 371 Date: Sep. 5, 2024

Related U.S. Application Data
(60) Provisional application No. 63/320,906, filed on Mar. 17, 2022.

(51) Int. Cl. G16H 50/20; C12Q 1/6806; C12Q 1/6869; C12Q 1/6886; G16B 20/20; G16B 40/20; G16H 20/10; G16H 50/70

(52) U.S. Cl. CPC G16H 50/20; C12Q 1/6806; C12Q 1/6869; C12Q 1/6886; G16B 20/20; G16B 40/20; G16H 20/10; G16H 50/70; C12Q 2600/106

(57) ABSTRACT

The present disclosure provides methods and systems that utilize analysis of cell-free DNA (cfDNA) fragmentation profiles in a sample obtained from a patient, determining a ratio of short to long fragments and a fragment size distribution from the fragmentation profile and determining a divergence score based on the ratio of short to long fragments in the sample as correlated to a ratio from a sample from a healthy patient, and determining, by the machine learning model, a monitoring score for the sample based on the fragmentation score, the divergence score, and the model weights, the monitoring score being indicative of a level of a tumor-derived nucleic acid in the cfDNA of the sample to detect, monitor, diagnose, predict cancer status, determine the likelihood of the presence of cancer and administer treatment to the patient.

### CROSS REFERENCE TO RELATED APPLICATIONS

This application claims the benefit of U.S. Provisional Patent Application Ser. No. 63/320,906 filed on Mar. 17, 2022, the entire content of which is incorporated herein by reference in its entirety.

### BACKGROUND OF THE INVENTION

### Field of the Invention

The invention relates generally to genetic analysis and more specifically to a method and system for analysis of cell-free DNA (cfDNA) fragments to predict the fraction of tumor-derived DNA modules (ctDNA burden) and detect cancer in a subject.

### Background Information

Much of the morbidity and mortality of human cancers world-wide is a result of the late diagnosis of these diseases, where treatments are less effective. Additionally, once cancer is diagnosed, predicting cancer progression and patient response to treatment is challenging which further undermines the success rate of cancer treatments. Unfortunately, clinically proven biomarkers that can be used to broadly diagnose and predict effective treatments for patients with cancer are not widely available.

The fraction of tumor-derived DNA molecules in the plasma (ctDNA burden) is a useful tool for describing the overall tumor burden in patients with cancer. Previous work has shown, the ctDNA burden in an individual patient is affected by many factors including the tumor's tissue of origin and stage as well as vascularization and perfusion. Accordingly, patients with later stage cancers have higher ctDNA burden than patients with earlier stage cancers. Similarly, patients with cancers in tissues with high cell turnover and direct access to the bloodstream (such as colorectal cancers) often have higher ctDNA burden than slower-growing tumors that are less vascular. The ctDNA burden may change over time as a tumor is exposed to treatment and dies (lowers) and subsequently acquires resistance mechanisms to the treatment and grows (raises). However, previous studies have lacked the ability to efficiently predict ctDNA burden and leverage the predicted ctDNA burden as a tool for diagnosing cancer, predicting disease progression and treatment response, and determining overall survival of a patient diagnosed with cancer.

### SUMMARY OF THE INVENTION

The present disclosure provides methods and systems that utilize analysis of cfDNA to monitor cancer progression and predict overall survival of a subject by scoring a cfDNA fragmentation profile obtained by analysis of cfDNA fragments in a sample obtained from the subject. The scoring methodology generates features that may be used to train a machine learning model to predict biomarkers that may be used to monitor cancer progression, evaluate patient responses to treatment, and predict the overall survivability of the subject.

As such, in one embodiment, the present invention provides a method of monitoring cancer. The method includes:
determining a cell-free DNA (cfDNA) fragmentation profile of a sample from a subject;
calculating a score based on the cfDNA fragmentation profile, the score being indicative of a likelihood of presence of cancer in the subject;
determining a ratio of short to long fragments and a fragment size distribution from the fragmentation profile;
calculating a divergence score based on the ratio of short to long fragments;
determining a set of model weights based on a fragment size distribution;
training a machine learning model using a set of features extracted from a plurality of fragmentation profiles of multiple subjects; and
determining, by the machine learning model, a monitoring score for the sample based on the monitoring score, the divergence score, and the model weights, the monitoring score being indicative of a level of a tumor-derived nucleic acid in the cfDNA of the sample.

In some aspects, the cfDNA fragmentation profile is determined by: cfDNA fragmentation profile is determined by: obtaining and isolating cfDNA fragments from the subject; sequencing the cfDNA fragments to obtain sequenced fragments; mapping the sequenced fragments to a genome to obtain windows of mapped sequences; and analyzing the windows of mapped sequences to determine cfDNA fragment lengths and generate the cfDNA fragmentation profile.

In another embodiment, the present invention provides a method of determining at least one of an overall survival a progression free survival, or a time to progression of a subject having cancer comprising. The method includes:
determining a cell-free DNA (cfDNA) fragmentation profile of a sample from the subject;
calculating a score based on the cfDNA fragmentation profile, the score being indicative of a likelihood of presence of cancer in the subject;
determining a ratio of short to long fragments and a fragment size distribution from the fragmentation profile;
calculating a divergence score based on the ratio of short to long fragments;
determining a set of model weights based on a fragment size distribution;
training a machine learning model using a set of features extracted from fragmentation profiles of multiple subjects;
determining, by the machine learning model, a monitoring score for the sample based on the score, the divergence score, and the model weights, the monitoring score being indicative of a level of a tumor-derived nucleic acid in cfDNA of the sample, thereby indicating a likelihood of cancer progression in the subject; and
determining at least one of a likelihood of overall survival, a progression free survival, or a time to progression of the subject based on the monitoring score, thereby determining overall survival of the subject. In yet another aspect, the present invention provides a method of treating a subject having cancer.

In still another embodiment, the present invention provides a system for monitoring cancer in a subject. The system includes:
a memory; and
one or more processors coupled to the memory, the one or more processors configured to perform operations that cause the computer system to:
determine a cell-free DNA (cfDNA) fragmentation profile of a sample from the subject;
calculate a score based on the cfDNA fragmentation profile, the score being indicative of a likelihood of presence of cancer in the subject;
determine a ratio of short to long fragments and a fragment size distribution from the fragmentation profile;
calculate a divergence score based on the ratio of short to long fragments;
determine a set of model weights based on a fragment size distribution;
train a machine learning model using a set of features extracted from fragmentation profiles of multiple subjects; and
determine, by the machine learning model, a monitoring score for the sample based on the score, the divergence score, and the model weights, the monitoring score being indicative of a level of a tumor-derived nucleic acid in cfDNA of the sample, thereby indicating a likelihood of cancer progression in the subject.

In another embodiment, the invention provides a non-transitory computer readable storage medium encoded with a computer program. The computer program includes instructions that when executed by one or more processors cause the one or more processors to perform operations to perform a method of the invention.

In yet another embodiment, the invention provides a computing system. The system includes a memory, and one or more processors coupled to the memory, with the one or more processors being configured to perform operations that implement a method of the invention.

In yet another embodiment, the invention provides a system for genetic analysis and assessing cancer that includes: (a) a sequencer configured to generate a whole genome sequencing (WGS) data set for a sample; and (b) a non-transitory computer readable storage medium and/or a computer system of the invention.

### BRIEF DESCRIPTION OF THE FIGURES

FIG. 1 is a block diagram illustrating a process for training a machine learning model to generate a DELFI monitoring score (DMS). The DMS may be used to monitor cancer, determine an overall survival, determine a progression free survival, and determine a time to progression of a patient diagnosed with cancer. The DMS may also be used to diagnose cancer in a patient and determine a cancer treatment to administer to a patient.

FIG. 2 is a graphical plot illustrating a comparison between observed MAF trajectories and trajectories determined based on DMS values.

FIG. 3 is a graphical plot illustrating a comparison between observed MAF trajectories and trajectories determined based on DMS values with no reference to timepoint or from which patient a given sample originated.

FIG. 4 is a graphical plot illustrating a comparison between observed MAF trajectories and trajectories determined based on DMS values. The cohort of patients having the observed MAF trajectories in this plot have a different type of cancer than the cohort of patients used to train the model generating the DMS values.

FIG. 5 is a graphical plot showing the progression free survival data by predicted DMS for two different cohorts having different types of cancer.

FIG. 6 is a graphical plot showing overall survival of a cohort of cancer patients using the predicted DMS at a pre-treatment time point. The graphical plot also shows overall survival of a cohort of cancer patients with incomplete and complete resections.

FIG. 7 is a graphical plot showing progression free survival of a cohort of cancer patients whose clonal variant MAF was bested to be 0% at the first post-treatment assessment. The graphical plot also shows a comparison between the predicted DMS and MAF by ddPCR.

FIG. 8 is an example computer 800 that may be used to implement the training algorithm show in FIG. 1 and generate DMS values.

FIGS. 9A-9B study design and patient flow diagram. FIG. 9A is a CAIRO5 study design flowchart. Once eligibility was confirmed, including the unresectable status of liver metastases as defined by the central panel, KRAS (exon 2, 3, and 4), NRAS (exon 2 and 3), and BRAF mutation status were assessed in tissue samples. Patients with RAS/BRAF mutant tumors were randomized between doublet chemotherapy plus bevacizumab (Arm 1) or triple chemotherapy plus bevacizumab (Arm 2). Patients with RAS/BRAF wild-type tumors were randomized between doublet chemotherapy plus either bevacizumab (Arm 3) or panitumumab (Arm 4). In the present translational study, blood draws from patients in Arm 1 (Mutant Arm) and Arm 3 (Wild-type Arm) were processed and analyzed. FIG. 9B is a Patient flow diagram. The number of patients and samples included in the study and the reasons for exclusion are depicted.

FIGS. 10A-10D illustrates the DELFI Tumor Fraction (DELFI-TF) is a mutation-agnostic approach for metastatic disease monitoring. FIG. 10A shows Patients with treatment-naïve non-operable liver-only mCRC enrolled in the CAIRO5 phase III trial had their tumors tested for hotspot mutations in KRAS, NRAS, and BRAF. Blood samples were collected at baseline, during treatment, and at the time of disease progression or last follow-up. Patients with driver mutations were monitored with ddPCR and DELFI-TF assays. Patients with wild-type KRAS, NRAS, and BRAF were monitored with DELFI-TF only. FIG. 10B shows plasma aliquots from patients with tissue-confirmed RAS/BRAF mutant mCRC were used for cfDNA isolation. From each time point, duplicate cfDNA samples were utilized for ddPCR and low-coverage WGS. WGS fragment-sequencing statistics were calculated per each sample at a given time point. A Bayesian probabilistic model was trained against the MAFs called by ddPCR readouts of the tumor-specific RAS/BRAF variants in all longitudinal cfDNA samples to generate the DELFI-TF values. FIG. 10C is a heatmap representation of genomic features depicts deviations of cfDNA fragment ratios and chromosomal arm-level z-scores across baseline and on-treatment time points of 128 patients, along with DELFI-TF values and clinical and demographic characteristics. FIG. 10D shows cfDNA genome-wide fragmentation profiles in 504 non-overlapping 5-Mb genomic regions at baseline and at time points near the second imaging assessment by RECIST1.1 show marked heterogeneity at baseline and for patients who exhibited disease progression compared to patients who experienced stable disease or radiologic response after initiating first-line systemic therapy.

FIGS. 11A-11F shows how DELFI-TF predicts tumor fraction in the blood of patients with advanced disease receiving systemic treatment. FIG. 11A shows patients with mCRC exhibit a wider range of DELFI-TF values at baseline than non-cancer controls. The 95% CI upper limit in non-cancer controls (gray dotted line) represents the DELFI-TF limit of blank (0.006). FIG. 11B shows that DELFI-TF strongly correlates with MAF measured by ddPCR across all study time points (n=692, Pearson correlation, r=0.85, p=3.9c-89). Plasma time points with undetectable MAF (n=60, red) exhibited a wide range of DELFI-TF values. FIG. 11C shows cfDNA fragmentation profiles (bottom) in a patient with wild-type mCRC exhibit short-to-long ratio aberrations even in the context of tumor copy neutral regions across 100-kb bins in a matched tissue sample (top). FIG. 11D shows Plasma tumor fractions assessed by DELFI-TF (blue) and RAS/BRAF MAF (orange) values correlate with cfDNA copy number changes in genomic regions that harbor frequently deleted (MBD1) or amplified (PLGC1) genes in colorectal cancer. FIG. 11E and FIG. 11F show relative coverages at the TSS positions for the group of 890 genes highly expressed in colorectal cancer show significantly deeper valleys for baseline samples (brown) than for on-treatment samples (purple) (Wilcoxon test, p<0.001), denoting, on average, lower tumor fractions at time points after treatment initiation.

FIGS. 12A-12C shows cfDNA tumor fraction assessed through whole-genome sequencing approaches. FIG. 12A shows patients with metastatic colorectal cancer exhibit a wider range of ichorCNA values at baseline compared to non-cancer controls. The 95% confidence interval upper limit in non-cancer controls is 0.017 (gray dotted line). FIG. 12B shows that DELFI-TF correlates to tumor fractions measured by ichorCNA for time points with undetectable mutant allele fractions for RAS/BRAF assessed by ddPCR in the mutant arm of the study (Spearman correlation, rho=0.54, p<0.001). FIG. 12C shows relative coverages at the transcription start site (TSS) positions for the group of genes highly expressed in colorectal cancer at baseline (brown), during systemic treatment (purple), after metastases resection (red), and at the time of disease relapse (black) for patient 65. Variations in valley depths reflect dynamic changes of plasma tumor fractions at longitudinal time points.

FIGS. 13A-13G shows that DELFI-TF is a non-invasive biomarker for metastatic disease burden and systemic treatment response. FIG. 13A shows that DELFI-TF (blue) and RAS/BRAF MAF (orange) values at baseline exhibit a moderate correlation with the SLDs of liver target lesions in pre-treatment imaging scans (DELFI-TF Spearman correlation, rho=0.49, p<0.001; MAF Spearman correlation, rho=0.48, p<0.001). FIG. 13B shows that DELFI-TF (blue) and RAS/BRAF MAF (orange) values at baseline exhibit no significant correlation with baseline levels of carcinoembryonic antigen (DELFI-TF Spearman correlation, rho=0.10, p=0.427; MAF Spearman correlation, rho=0.15, p<0.236). FIG. 13C shows that DELFI-TF and MAF values at baseline were significantly lower in patients with a later confirmed partial response (PR) or complete response (CR) by two consecutive RECIST 1.1 measurements (DELFI-TF, Wilcoxon test, p=0.048; MAF, Wilcoxon test, p=0.017). FIG. 13D shows that DELFI-TF and MAF values at baseline were significantly lower in patients surgically treated with complete resection (orange) or partial resection (green) after systemic treatment (DELFI-TF, Kruskal-Wallis test, p=0.037; MAF, Kruskal-Wallis test, p=0.011). FIG. 13E shows that colorectal cancer patients with metachronous metastases (gray) exhibit lower tumor fractions assessed by DELFI-TF at baseline than patients who presented with synchronous metastases (green) (Wilcoxon test, p<0.001). FIG. 13F shows that liver metastases are highlighted by blue circles in longitudinal imaging scans (top). cfDNA tumor fraction dynamics (DELFI-TF, MAF) and the SLD values are shown for study patient 11 (bottom). Treatments are indicated by shaded bars. The purple dotted line represents the time for primary tumor resection a few weeks after liver metastases removal. DELFI-TF and ddPCR MAF for the KRAS G12D mutation accurately track disease burden dynamics before and after the complete resection. FIG. 13G shows that patients who eventually experienced disease progression (top) more often exhibited increased DELFI-TF values at longitudinal time points than patients who never presented with disease progression (bottom).

FIGS. 14A-14D shows DELFI-TF and colorectal cancer clinical features. FIG. 14A shows tumor fractions assessed by DELFI-TF and MAF at baseline are equivalent between colon cancers from left (brown) or right (beige) sides (DELFI-TF, Wilcoxon test, p=0.329; MAF, Wilcoxon test, p=0.515). FIG. 14B shows tumor fractions assessed by DELFI-TF at baseline are equivalent between colon cancers with driver RAS/BRAF mutations (red) or wild-type (pink) (Wilcoxon test, p=1). FIG. 14C shows tumor fractions assessed by DELFI-TF and MAF at baseline are higher in patients who eventually experienced disease progression (green) than patients who never experienced disease progression (red) (DELFI-TF, Wilcoxon test, p=0.03; MAF, Wilcoxon test, p=0.02). The sum of the largest diameters at baseline did not differ between ever progressors (green) and never progressors (red) (Wilcoxon test, p=0.94) FIG. 14D shows a waterfall plot that depicts objective clinical response through the sum of the longest diameters (SLD) changes. Red, DELFI-TF slope above the median. Cyan, DELFI-TF slopes below the median.

FIGS. 15A-15B shows DELFI-TF dynamics in study patients. FIG. 15A shows RAS/BRAF wild-type arm. FIG. 15B RAS/BRAF mutant arm.

FIG. 16 shows that Dynamic changes in DELFI-TF are associated with longitudinal clinical outcomes. DELFI-TF slopes were calculated using all time points available for patients with at least a baseline and one blood draw within 60 days of disease progression (n=81). Left, DELFI-TF slopes are colored based on results below (cyan) or above (red) the median DELFI-TF slope. Right, swimmer plot encompassing RECIST 1.1, liquid biopsy, surgery, and death events for patients ordered according to time on the study in weeks. Each bar represents the interval between the study registration and death or last follow-up. Bar segments are colored according to the RECIST 1.1 readouts.

FIGS. 17A-17D shows baseline DELFI-TF and DELFI-TF slopes correlate with progression-free survival (PFS) and overall survival (OS). FIG. 17A shows Kaplan-Meier curves for PFS according to baseline DELFI-TF values below (orange) or above (blue) the lower quartile among patients with RAS/BRAF mutant and wild-type metastatic colorectal cancer (n=128) (Log-Rank p=0.015). FIG. 17B shows Kaplan-Meier curves for PFS according to DELFI-TF slopes below (orange) or above (blue) the median among patients with at least one blood draw within 60 days of disease progression (n=81) (Log-Rank p<0.001). FIG. 17C shows Kaplan-Meier curves for PFS among patients who experienced imaging response or stable disease that lasted longer than 12 months (durable clinical benefit) according to DELFI-TF slopes below (orange) or above (blue) the median (n=42) (Log-Rank p<0.001). FIG. 17D shows Kaplan-Meier curves for OS according to DELFI-TF slopes below (orange) or above (blue) the median among patients with at least one blood draw within 60 days of disease progression (n=81) (Log-Rank p<0.001).

FIGS. 18A-18D shows imaging and plasma biomarkers for survival outcomes in metastatic colorectal cancer patients. FIG. 18A shows Kaplan-Meier curves for progression-free survival (PFS) according to imaging response assessed by RECIST 1.1 show no survival difference between patients with partial response (PR) (orange) or stable disease (SD) (blue). PD, progressive disease. FIG. 18B shows Kaplan-Meier curves for PFS according to baseline MAF for RAS/BRAF below (orange) or above (blue) the lower quartile value among patients with RAS/BRAF mutant metastatic colorectal cancer (n=65) (Log-Rank p=0.003). FIG. 18C shows Kaplan-Meier curves for PFS according to baseline carcinoembryonic antigen values below (orange) or above (blue) the lower quartile value among patients with metastatic colorectal cancer (n=127) (Log-Rank p=0.067). FIG. 18D shows Kaplan-Meier curves for overall survival (OS) according to surgical status and DELFI-TF slope among patients with at least one blood draw within 60 days of disease progression (n=81) (Log-Rank p<0.001).

### DETAILED DESCRIPTION OF THE INVENTION

Described herein is a non-invasive method for monitoring cancer, as well as prediction of overall survival, progression free survival, and time to progression of a subject having cancer. cfDNA in the blood can provide a non-invasive way to monitor disease for patients with cancer. As demonstrated herein, DNA Evaluation of Fragments for early Interception (DELFI) was used to evaluate genome-wide fragmentation patterns of cfDNA of patients with various types of cancers, as well as healthy individuals. Evaluation of cfDNA included a scoring methodology. A defined score (also referred to herein as ‘DELFI monitoring score’) was determined based cfDNA fragmentation profiles obtained using cfDNA fragments of a given patient sample. Assessing cfDNA using the methodology described herein can also provide an approach for monitoring cancer, which can increase the chance for successful treatment and improved outcome of a patient having cancer.

Before the present compositions and methods are described, it is to be understood that this invention is not limited to the particular methods and systems described, as such methods and systems may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The present disclosure provides innovative methods and systems for analysis of cfDNA to monitor, detect, or otherwise assess cancer. As indicated in prior studies, on average, cancer-free individuals have longer cfDNA fragments (average size of 167.09 bp) whereas individuals with cancer have shorter cfDNA fragments (average size of 164.88 bp). The methodology described herein allows simultaneous analysis of a large number of abnormalities in cfDNA through genome-wide analysis of cfDNA fragmentation patterns.

As such, in one embodiment, the present invention provides a method of monitor cancer in a subject. The method includes:
determining a cell-free DNA (cfDNA) fragmentation profile of a sample from a subject;
calculating a score based on the cfDNA fragmentation profile, the score being indicative of a likelihood of presence of cancer in the subject;
determining a ratio of short to long fragments and a fragment size distribution from the fragmentation profile;
calculating a divergence score based on the ratio of short to long fragments;
determining a set of model weights based on a fragment size distribution;
training a machine learning model using a set of features extracted from a plurality of fragmentation profiles of multiple subjects; and
determining, by the machine learning model, a monitoring score for the sample based on the monitoring score, the divergence score, and the model weights, the monitoring score being indicative of a level of a tumor-derived nucleic acid in the cfDNA of the sample.

In embodiment, the present invention provides a method of treating a subject having cancer. The method includes:
a) detecting cancer in the subject using the methodology of the invention, or determining overall survival of the subject using the methodology of the invention; and
b) administering a cancer treatment to the subject, thereby treating the subject.

In another embodiment, the present invention provides a method of monitoring cancer in a subject. The method includes:
a) detecting cancer in the subject using the methodology of the invention, or determining overall survival of the subject using the methodology of the invention;
b) administering a cancer treatment to the subject; and
c) determining overall survival of the subject using the methodology of the invention after the cancer treatment is administered, thereby monitoring cancer in the subject.

The methodology described herein utilizes cfDNA fragmentation profiles. As used herein, the terms “fragmentation profile,” In some aspects, determining a cfDNA fragmentation profile in a mammal can be used for identifying a mammal as having cancer. For example, cfDNA fragments obtained from a mammal (e.g., from a sample obtained from a mammal) can be subjected to low coverage whole-genome sequencing, and the sequenced fragments can be mapped to the genome (e.g., in non-overlapping windows) and assessed to determine a cfDNA fragmentation profile. A cfDNA fragmentation profile of a mammal having cancer is more heterogeneous (e.g., in fragment lengths) than a cfDNA fragmentation profile of a healthy mammal (e.g., a mammal not having cancer).

A cfDNA fragmentation profile can include one or more cfDNA fragmentation patterns. A cfDNA fragmentation pattern can include any appropriate cfDNA fragmentation pattern. Examples of cfDNA fragmentation patterns include, without limitation, fragment size density, median fragment size, fragment size distribution, ratio of small cfDNA fragments to large cfDNA fragments, and the coverage of cfDNA fragments. In some aspects, a cfDNA fragmentation profile can be a genome-wide cfDNA profile (e.g., a genome-wide cfDNA profile in windows across the genome). In some aspects, a cfDNA fragmentation profile can be a targeted region profile. A targeted region can be any appropriate portion of the genome (e.g., a chromosomal region). Examples of chromosomal regions for which a cfDNA fragmentation profile can be determined as described herein include, without limitation, a portion of a chromosome (e.g., a portion of 2 q, 4 p, 5 p, 6 q, 7 p, 8 q, 9 q, 10 q, 11 q, 12 q, and/or 14 q) and a chromosomal arm (e.g., a chromosomal arm of 8 q, 13 q, 11 q, and/or 3 p). In some cases, a cfDNA fragmentation profile can include two or more targeted region profiles.

In various aspects, cfDNA obtained from a sample is isolated and fragments of a particular size range are utilized in analysis. In some aspects, analyzing excludes fragment sizes less than about 10, 50, 100 or 105 bp and greater than about 220, 250, 300, 350 bp or more. In some aspects, analyzing excludes fragment sizes less than 105 bp and greater than 170 bp. In some aspects, analyzing excludes fragment sizes less than about 230, 240, 250, 260 bp and greater than about 420, 430, 440, 450 bp or greater. In some aspects, analyzing excludes fragment sizes less than 260 bp and greater than 440 bp.

In some aspects, a cfDNA fragmentation profile may be being determined by: processing a sample from the subject comprising cfDNA fragments into sequencing libraries; subjecting the sequencing libraries to low-coverage whole genome sequencing to obtain sequenced fragments; mapping the sequenced fragments to a genome to obtain windows of mapped sequences; and analyzing the windows of mapped sequences to determine cfDNA fragment lengths.

In some aspects, a cfDNA fragmentation profile may be being determined by: obtaining and isolating cfDNA fragments from the subject, sequencing the cfDNA fragments to obtain sequenced fragments, mapping the sequenced fragments to a genome to obtain windows of mapped sequences, and analyzing the windows of mapped sequences to determine cfDNA fragment lengths and generate the cfDNA fragmentation profile.

The methodology of the present invention is based on low coverage whole genome sequencing and analysis of isolated cfDNA. In one aspect, the data used to develop the methodology of the invention is based on shallow whole genome sequence data (1-2× coverage).

In some aspects, mapped sequences are analyzed in non-overlapping windows covering the genome. Conceptually, windows may range in size from thousands to millions of bases, resulting in hundreds to thousands of windows in the genome. 5 Mb windows were used for evaluating cfDNA fragmentation patterns as these would provide over 20,000 reads per window even at a limited amount of 1-2× genome coverage. Within each window, the coverage and size distribution of cfDNA fragments was examined. In some aspects, the genome-wide pattern from an individual can be compared to reference populations to determine if the pattern is likely healthy or cancer-derived.

In certain aspects, the mapped sequences include tens to thousands of genomic windows, such as 10, 50, 100 to 1,000, 5,000, 10,000 or more windows. Such windows may be non-overlapping or overlapping and include about 1, 2, 3, 4, 5, 6, 7, 8, 9 or 10 million base pairs.

In various aspects, a cfDNA fragmentation profile is determined within each window. As such, the invention provides methods for determining a cfDNA fragmentation profile in a subject (e.g., in a sample obtained from a subject).

In some aspects, a cfDNA fragmentation profile can be used to identify changes (e.g., alterations) in cfDNA fragment lengths. An alteration can be a genome-wide alteration or an alteration in one or more targeted regions/loci. A target region can be any region containing one or more cancer-specific alterations. In some aspects, a cfDNA fragmentation profile can be used to identify (e.g., simultaneously identify) from about 10 alterations to about 500 alterations (e.g., from about 25 to about 500, from about 50 to about 500, from about 100 to about 500, from about 200 to about 500, from about 300 to about 500, from about 10 to about 400, from about 10 to about 300, from about 10 to about 200, from about 10 to about 100, from about 10 to about 50, from about 20 to about 400, from about 30 to about 300, from about 40 to about 200, from about 50 to about 100, from about 20 to about 100, from about 25 to about 75, from about 50 to about 250, or from about 100 to about 200, alterations).

In various aspects, a cfDNA fragmentation profile can include a cfDNA fragment size pattern. cfDNA fragments can be any appropriate size. For example, in some aspects, a cfDNA fragment can be from about 50 base pairs (bp) to about 400 bp in length. As described herein, a subject having cancer can have a cfDNA fragment size pattern that contains a shorter median cfDNA fragment size than the median cfDNA fragment size in a healthy subject. A healthy subject (e.g., a subject not having cancer) can have cfDNA fragment sizes having a median cfDNA fragment size from about 166.6 bp to about 167.2 bp (e.g., about 166.9 bp). In some aspects, a subject having cancer can have cfDNA fragment sizes that are, on average, about 1.28 bp to about 2.49 bp (e.g., about 1.88 bp) shorter than cfDNA fragment sizes in a healthy subject. For example, a subject having cancer can have cfDNA fragment sizes having a median cfDNA fragment size of about 164.11 bp to about 165.92 bp (e.g., about 165.02 bp).

In some aspects, a dinucleosomal cfDNA fragment can be from about 230 base pairs (bp) to about 450 bp in length. As described herein, a subject having cancer can have a dinucleosomal cfDNA fragment size pattern that contains a shorter median dinucleosomal cfDNA fragment size than the median dinucleosomal cfDNA fragment size in a healthy subject. In some aspects, on average, cancer-free subjects have longer cfDNA fragments in the dinucleosomal range (average size of 334.75 bp) whereas subjects with cancer have shorter dinucleosomal cfDNA fragments (average size of 329.6 bp). As such, a healthy subject (e.g., a subject not having cancer) can have dinucleosomal cfDNA fragment sizes having a median cfDNA fragment size of about 334.75 bp. In some aspects, a subject having cancer can have dinucleosomal cfDNA fragment sizes that are shorter than dinucleosomal cfDNA fragment sizes in a healthy subject. For example, a subject having cancer can have dinucleosomal cfDNA fragment sizes having a median cfDNA fragment size of about 329.6 bp.

A cfDNA fragmentation profile can include a cfDNA fragment size distribution. As described herein, a subject having cancer can have a cfDNA size distribution that is more variable than a cfDNA fragment size distribution in a healthy subject. In some aspects, a size distribution can be within a targeted region. A healthy subject (e.g., a subject not having cancer) can have a targeted region cfDNA fragment size distribution of about 1 or less than about 1. In some aspects, a subject having cancer can have a targeted region cfDNA fragment size distribution that is longer (e.g., 10, 15, 20, 25, 30, 35, 40, 45, 50 or more bp longer, or any number of base pairs between these numbers) than a targeted region cfDNA fragment size distribution in a healthy subject. In some aspects, a subject having cancer can have a targeted region cfDNA fragment size distribution that is shorter (e.g., 10, 15, 20, 25, 30, 35, 40, 45, 50 or more bp shorter, or any number of base pairs between these numbers) than a targeted region cfDNA fragment size distribution in a healthy subject. In some aspects, a subject having cancer can have a targeted region cfDNA fragment size distribution that is about 47 bp smaller to about 30 bp longer than a targeted region cfDNA fragment size distribution in a healthy subject. In some aspects, a subject having cancer can have a targeted region cfDNA fragment size distribution of, on average, a 10, 11, 12, 13, 14, 15, 15, 17, 18, 19, 20 or more bp difference in lengths of cfDNA fragments. For example, a subject having cancer can have a targeted region cfDNA fragment size distribution of, on average, about a 13 bp difference in lengths of cfDNA fragments. In some aspects, a size distribution can be a genome-wide size distribution.

A cfDNA fragmentation profile can include a ratio of small cfDNA fragments to large cfDNA fragments and a correlation of fragment ratios to reference fragment ratios. As used herein, with respect to ratios of small cfDNA fragments to large cfDNA fragments, a small cfDNA fragment can be from about 100 bp in length to about 150 bp in length. As used herein, with respect to ratios of small cfDNA fragments to large cfDNA fragments, a large cfDNA fragment can be from about 151 bp in length to 220 bp in length. As described herein, a subject having cancer can have a correlation of fragment ratios (e.g., a correlation of cfDNA fragment ratios to reference DNA fragment ratios such as DNA fragment ratios from one or more healthy subjects) that is lower (e.g., 2-fold lower, 3-fold lower, 4-fold lower, 5-fold lower, 6-fold lower, 7-fold lower, 8-fold lower, 9-fold lower, 10-fold lower, or more) than in a healthy subject. A healthy subject (e.g., a subject not having cancer) can have a correlation of fragment ratios (e.g., a correlation of cfDNA fragment ratios to reference DNA fragment ratios such as DNA fragment ratios from one or more healthy subjects) of about 1 (e.g., about 0.96). In some aspects, a subject having cancer can have a correlation of fragment ratios (e.g., a correlation of cfDNA fragment ratios to reference DNA fragment ratios such as DNA fragment ratios from one or more healthy subjects) that is, on average, about 0.19 to about 0.30 (e.g., about 0.25) lower than a correlation of fragment ratios (e.g., a correlation of cfDNA fragment ratios to reference DNA fragment ratios such as DNA fragment ratios from one or more healthy subjects) in a healthy subject.

The methodology of the present invention further includes predicting a mutant allele fraction (MAF) based on a cfDNA fragmentation profile. The MAF of a mutation in DNA is a common value reported by diagnostic tests for oncology and represents the fraction of DNA molecules analyzed that contain the mutation of interest. For a tumor-derived variant identified in circulating, cell-free DNA (cfDNA), the MAF represents the fraction of all cfDNA that contains the variant. cfDNA is a combination of tumor-derived and normal cell-derived DNA, and as such, the MAF value of a clonal somatic variant captures the fraction of cfDNA that is tumor-derived. This MAF is correlated to, and can therefore be used as a proxy for, the circulating tumor DNA fraction.

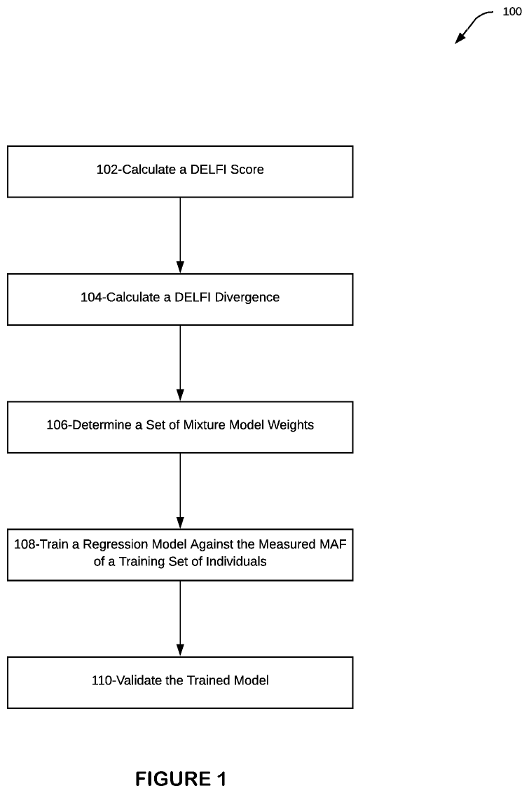

In various embodiments, the present invention may use the predicted MAF to detect cancer in a subject, predict disease prognosis, predict response to treatment, and/or assess overall survival of the subject. FIG. 1 is a block diagram illustrating a process for training a machine learning model to predict MAF. The predicted MAF may be referred to as the DELFI monitoring score (DMS). At 102, a DELFI score is calculated based on a cfDNA fragmentation profile. The DELFI score indicates how similar the fragmentation profile looks to an archetypal individual with cancer or an individual without cancer. In some aspects, calculating the DELFI score includes: i) determining a ratio of short to long cfDNA fragments of the sample, ii) determining a Z-score for cfDNA fragments of the sample by chromosome arm, iii) quantifying cfDNA fragment density using a computational mixture model analysis, and iv) using a machine learning model to process output of i)-iii) to define the score. In various aspects, the score is utilized to determine a likelihood of overall survival of the subject.

In one illustrative example (Example 1), in a multi-cancer cohort, the inventors calculated from low coverage whole genome sequencing the ratio of short to long fragments by 5 MB bins, Z-scores by chromosome arm, and a mixture model of cfDNA fragment sizes, for each individual. Using these features as input, the inventors fit a cross-validated gradient boosted machine to the cancer status of each person (Cancer/No Cancer). The output of this model is a score ranging from 0 to 1, with high numbers indicating a stronger signal of cancer and low numbers more similarity to non-cancer. The score generated using these techniques may be used a feature to training a machine learning model to generate a DMS.

At 104, a DELFI divergence may be calculated. In some aspects, the DELFI divergence may be equal to one minus the correlation between the binned and mean centered short to long ratios of a given sample and the binned and mean centered short to long ratios of a healthy sample. For example, the healthy sample may equal to the median value for the binned and mean centered short to long ratios of a reference cohort containing only healthy samples. As used herein, the mean centered short to long ratio is the binned short to long ratio minus the overall mean.

At 106, a set of weights may be determined for a computational mixture model. In some aspects, the mixture model may be a vector including 11 weights that summarize the fragmentation distribution in the sample. The weights from the mixture model are estimated using a Bayesian mixture of normal distributions of the empirical fragment size distribution.

At 108, a regression model may be trained against the measured MAF of individuals so that the model learns the features of a sample's DELFI Score, DELFI divergence, and mixture model weights that contribute to a known MAF for the sample. For example, the MAF may relate to the tumor burden, e.g., as estimated by MAF. In some aspects, the regression model may be a Bayesian Hierarchical Regression model that includes multiple layers with each layer including more predictors. At runtime, the model takes the DELFI Score, DELFI divergence, and mixture model weights as inputs and outputs a predicted MAF. Training is done via Leave-One-Patient-Out cross-validation. In this cross-validation scheme, each patient's data is held-out in turn, the model is trained on the remaining samples, and that trained model is then used to generate predictions for the held-out samples. In one example of the model, MAF is a beta-distributed random variable and the model assumes that the expected MAF of a given sample is functionally related to the described features via the inverse-logit of the feature-matrix multiplied by a vector of regression coefficients plus a patient-specific random intercept which accounts for within-patient correlation between measurements.

At 110, the trained model may be validated to confirm it achieves a desired level of accuracy. In various embodiments, the trained model may be evaluated statistically and clinically. For example, the quality of the generated predictions may be evaluated by assessing the correlation of the predicted tumor burden with the observed tumor burden values. Other examples of validation schemes performed to evaluate the trained model include observing longitudinal plots displaying the measured tumor burden values with the predicted tumor burden values overlaid and assessing the relationship between the tumor burden predictions and time-to-death in patients.

The model may also be validated in clinical settings to understand the clinical utility of the predictions. To clinically validate the model two treatment-naive metastatic cohorts were obtained. The cohorts were beginning antineoplastic treatment with chemotherapy or targeted agents and the ability of the predicted ctDNA burden (represented by MAF) to predict survival of each patient in the cohorts at the baseline and first post-treatment blood draws was determined. For 76 patients with metastatic colorectal cancer (mCRC) and 17 patients with metastatic non-small cell lung cancer (mNSCLC), observed MAF data was obtained. All of the patient samples were analyzed independently with the DELFI Monitoring Score approach. The first post-treatment blood draws were taken between 4-12 weeks and 1-3 weeks post-treatment for the mCRC and mNSCLC patients, respectively. MAF was measured for the clonal variants by digital droplet PCR (RAS/RAF variants) and deep targeted NGS sequencing (EGFR) for the mCRC and mNSCLC patients, respectively.

A Kaplan-Meier estimator was used to assess the predictive value of a single threshold for the modeled ctDNA burden. In some aspects, the threshold of the DMS may be chosen for each cohort via a leave-one-patient-out cross-validation. In this analysis, one sample was removed, and the threshold which minimized the log-rank p-value was selected. This process was repeated for each patient in the cohort, and the median of all optimized thresholds from the cross-validation was chosen as the final threshold for the Kaplan-Meier estimates. A Cox Proportional Hazard model was also used to assess the predictive value of the continuous modeled ctDNA burden for progression-free survival and overall survival, where available. In another aspect, other approaches to determine a threshold may be used, such as using a reference set of individuals with no or low tumor fraction.

### Model Validation Results

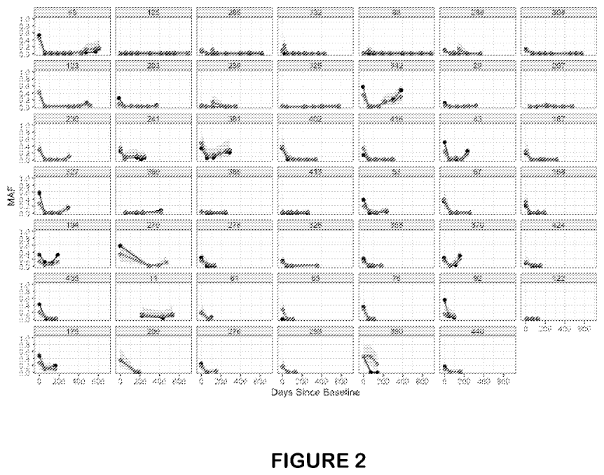

FIG. 2 is a graphical plot illustrating a comparison between observed longitudinal MAF data and the predicted tumor burden generated by the model. Specifically, the plots illustrate the relative MAF trajectories of mCRC patients obtained from observed and predicted MAF data. Each panel plots the observed longitudinal MAF data with black lines and the predictions from the model overlaid in blue lines. The plots also show the bounds of the 95% Bayesian credible interval overlaid as a light blue shaded region over the black and blue lines. Each panel represents longitudinal data from one subject and the observed trajectory of a given patient's MAF is closely matched by the predicted MAF (i.e., the DMS) in most cases.

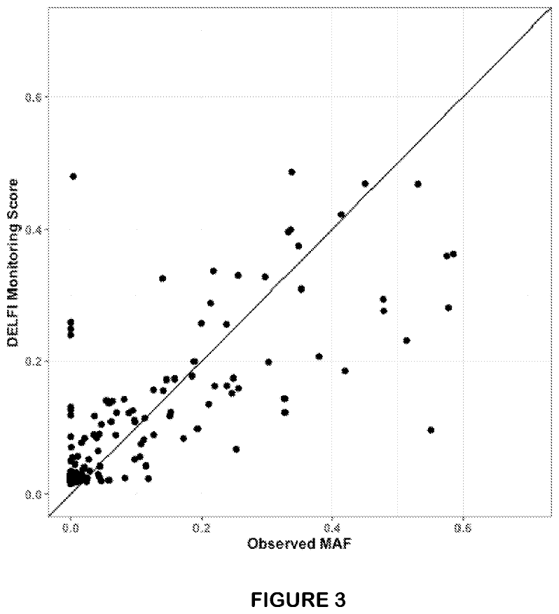

FIG. 3 is a graphical plot illustrating a comparison between the observed and predicted MAF without reference to a particular timepoint or from which patient a given sample originated. Accordingly, the plot includes the DMS and observed MAF for the entire mCRC cohort. The solid diagonal line included in the plot represents a line of equality between the DMS and observed MAF. As shown in the figure, the DELFI monitoring score matches the observed MAF fairly well in most cases. In the few instances where the predictions are far from the line of equality, there is evidence that, especially in samples in which the measured MAF is low (i.e. less than 1%), the issue is not with the model but with the measurement process. For example, due to tumor and/or metastases heterogeneity and clonal evolution that may occur upon treatment, a variant of interest may be more difficult to assess.

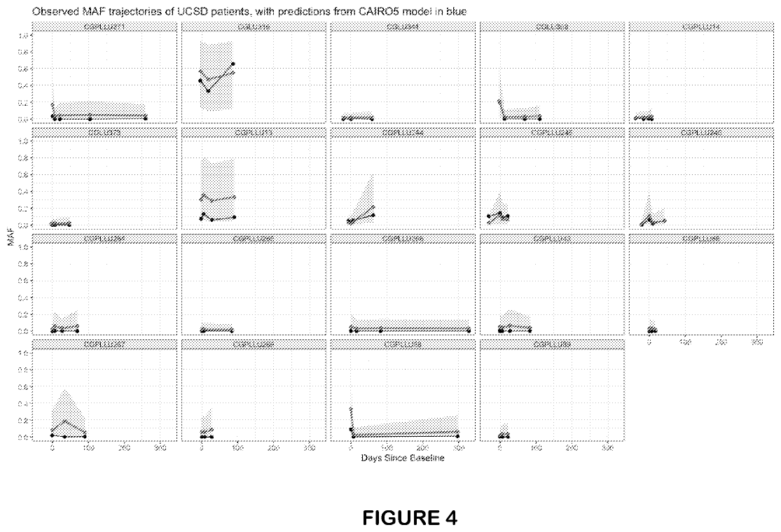

FIG. 4 is a graphical plot illustrating a comparison between observed MAF trajectories of mNSCLC patients and DMS values from the model trained on the mCRC cohort. Each panel plots the observed longitudinal MAF data with black lines and the predictions from the model overlaid in blue lines. The plots also show the bounds of the 95% Bayesian credible interval overlaid as a light blue shaded region over the black and blue lines. Each panel represents longitudinal data from one subject and the observed trajectory of a given patient's MAF is closely matched by the predicted MAF (i.e., the DMS) in most cases.

The results of the comparison indicate that the model trained on MAF data from patients having one type of cancer (e.g., the mCRC patients) may be successfully applied to the patients having a different type of cancer (e.g., the mNSCLC cohort). The external applicability is a desirable feature of predictive models, as the predictions are of generally high quality despite the substantive differences between the two cohorts (cancer type, sequencing depth, etc.). The external applicability of the predication model described herein improves the efficiency of prediction model development and training by enabling one prediction model trained on a specific data set to be used to generate useful predictions for patients that are different than the patients included in the training dataset.

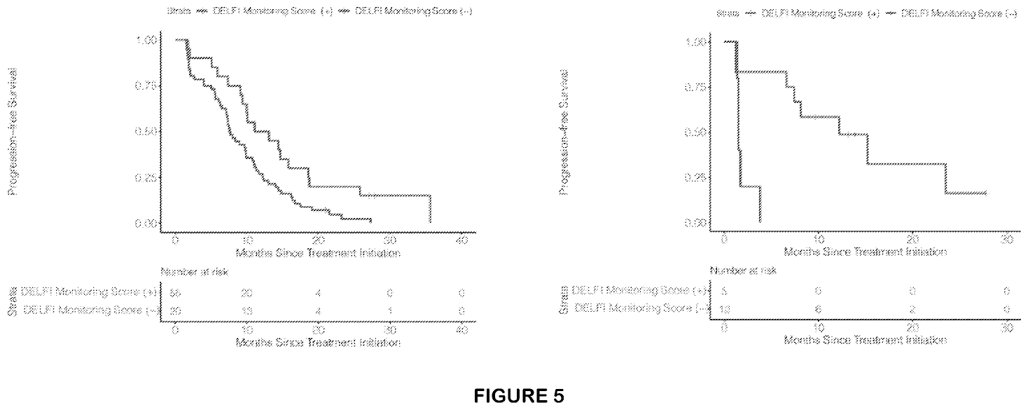

FIG. 5 is a graphical plot illustrating the progression-free survival in a metastatic colorectal cancer cohort (i.e., the mCRC cohort) in panel A and the progression free survival in a metastatic lung cancer cohort (i.e., the mNSCLC cohort) in panel B. The plot illustrates the results from the Kaplan-Meier estimation that uses the cohort-specific cross-validated thresholds to distinguish patients with high and low DMS. In both cohorts, patients whose first timepoint post-treatment had a DMS below the cross-validated threshold (DELFI Monitoring Score (-)) showed longer progression-free survival than patients with high DMS. These results indicate the DMS can distinguish subgroups of patients who are more likely to survive without progression as soon as the first post-treatment timepoint.

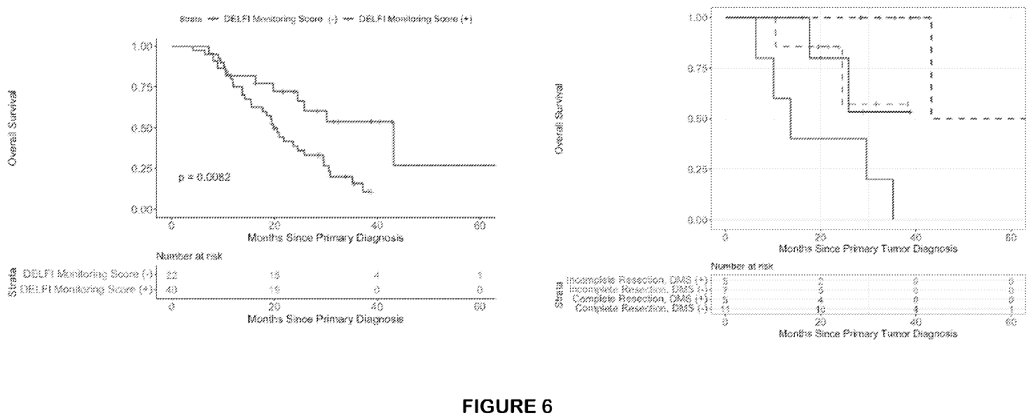

FIG. 6 is a plot illustrating the overall survival of the mCRC cohort based on the DMS in panel A and the overall survival of the mCRC after treatment based on the DMS in panel B. To determine the ability of DMS to predict response to treatment, the overall survival data for the mCRC cohort was obtained. The survival data for each patient was labeled with an indication of whether each patient who received surgery had a complete or incomplete resection. The DMS of the timepoint prior to treatment initiation was evaluated against a separate cross-validated distinguishing threshold. As shown in panel A, patients with DMS below the threshold had longer overall survival than those with DMS above the threshold. Additionally, in patients who received surgery, the DMS was able to further distinguish the overall survival for patients who had incomplete or complete resections in panel B with patients having complete resections having a longer predicted overall survival. Taken together, the results illustrate the DMS's feasibility to predict disease prognosis, even before treatment begins.

The MAF of clonal variants is correlated to the ctDNA burden and can therefore be useful as a quantitative metric for estimating the fraction of plasma DNA derived from the tumor and overall tumor burden in a patient. However, over the course of treatment, a tumor's genetic profile may change under the selective pressures of the treatment. Therefore, measuring the MAF of only one variant is limited for measuring patient response longitudinally. To evaluate the sensitivity of the DMS to changes in tumor DNA during treatment, it was determined if patients on treatment with MAF of the clonal variant measured to be 0% at the first post-treatment timepoint would benefit from an analysis with the DMS.

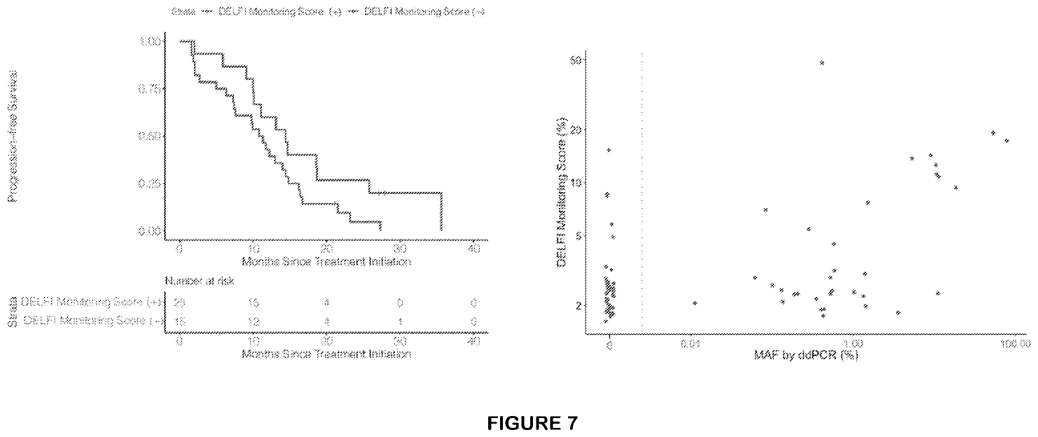

FIG. 7 is a plot illustrating the progression free survival of patients in the mCRC cohort whose clonal variant MAF was measured to be 0% at the first post-treatment assessment in panel A and the DMS vs MAF by ddPCR of the patients in the mCRC cohort whose clonal variant MAF was measured to be 0% at the first post-treatment in panel B. As shown in panel A, the 43 patients with 0% MAF in the mCRC cohort were further separated by the DMS with respect to progression-free survival. Some of these patients measured between 5%-15% DMS, as shown in panel B. This data indicates that the predicted ctDNA burden based on genome-wide assessment of fragments may be more sensitive than conventional MAF.

Additionally, a Cox Proportional Hazards analysis was performed on the mCRC and mNSCLC cohorts to evaluate the predictive value of the continuous DMS. At both the pre-treatment and first post-treatment timepoints, the DMS was predictive for overall survival in the mCRC cohort (HR: 19.2, 95% CI: 2.7-138.5 and HR: 400.4, 95% CI: 11.8-13581.0, respectively) and progression-free survival in the mNSCLC cohort (HR: 67.3, 95% CI: 1.1-4073.6 and HR: 246.5, 95% CI: 2.2-28030.9, respectively).

### Example Hardware Implementation

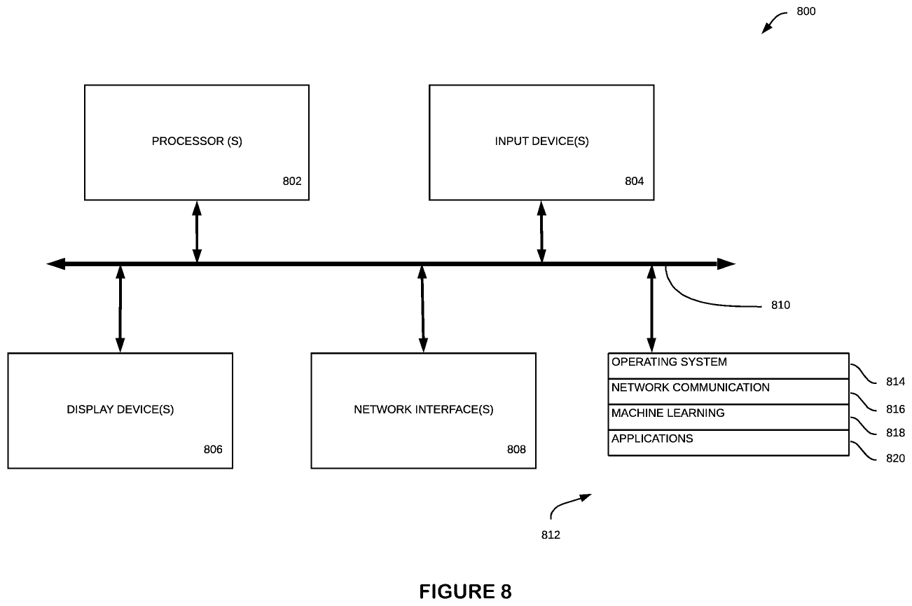

FIG. 8 illustrates an example computer 800 that may be used to implement the training algorithm show in FIG. 1 and generate DMS values. For example, the computer 800 may include a machine learning system that trains a machine learning model to generate DMS values as described above or a portion or combination thereof in some embodiments. The computer 800 may be any electronic device that runs software applications derived from compiled instructions, including without limitation personal computers, servers, smart phones, media players, electronic tablets, game consoles, email devices, etc. In some implementations, the computer 800 may include one or more processors 802, one or more input devices 804, one or more display devices 806, one or more network interfaces 808, and one or more computer-readable mediums 812. Each of these components may be coupled by bus 810, and in some embodiments, these components may be distributed among multiple physical locations and coupled by a network.

Display device 806 may be any known display technology, including but not limited to display devices using Liquid Crystal Display (LCD) or Light Emitting Diode (LED) technology. Processor(s) 802 may use any known processor technology, including but not limited to graphics processors and multi-core processors. Input device 804 may be any known input device technology, including but not limited to a keyboard (including a virtual keyboard), mouse, track ball, camera, and touch-sensitive pad or display. Bus 810 may be any known internal or external bus technology, including but not limited to ISA, EISA, PCI, PCI Express, USB, Serial ATA or Fire Wire. Computer-readable medium 812 may be any non-transitory medium that participates in providing instructions to processor(s) 804 for execution, including without limitation, non-volatile storage media (e.g., optical disks, magnetic disks, flash drives, etc.), or volatile media (e.g., SDRAM, ROM, etc.).

Computer-readable medium 812 may include various instructions 814 for implementing an operating system (e.g., Mac OS®, Windows®, Linux). The operating system may be multi-user, multiprocessing, multitasking, multithreading, real-time, and the like. The operating system may perform basic tasks, including but not limited to: recognizing input from input device 804; sending output to display device 806; keeping track of files and directories on computer-readable medium 812; controlling peripheral devices (e.g., disk drives, printers, etc.) which can be controlled directly or through an I/O controller; and managing traffic on bus 810. Network communications instructions 816 may establish and maintain network connections (e.g., software for implementing communication protocols, such as TCP/IP, HTTP, Ethernet, telephony, etc.).

Machine learning instructions 818 may include instructions that enable computer 800 to function as a machine learning system and/or to training machine learning models to generate DMS values as described herein. Application(s) 820 may be an application that uses or implements the processes described herein and/or other processes. The processes may also be implemented in operating system 814. For example, application 820 and/or operating system may create tasks in applications as described herein.

The described features may be implemented in one or more computer programs that may be executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program may be written in any form of programming language (e.g., Objective-C, Java), including compiled or interpreted languages, and it may be deployed in any form, including as a stand-alone program or as a module, component, subroutine, or other unit suitable for use in a computing environment.

Suitable processors for the execution of a program of instructions may include, by way of example, both general and special purpose microprocessors, and the sole processor or one of multiple processors or cores, of any kind of computer. Generally, a processor may receive instructions and data from a read-only memory or a random access memory or both. The essential elements of a computer may include a processor for executing instructions and one or more memories for storing instructions and data. Generally, a computer may also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory may be supplemented by, or incorporated in, ASICs (application-specific integrated circuits).

To provide for interaction with a user, the features may be implemented on a computer having a display device such as an LED or LCD monitor for displaying information to the user and a keyboard and a pointing device such as a mouse or a trackball by which the user can provide input to the computer.

The features may be implemented in a computer system that includes a back-end component, such as a data server, or that includes a middleware component, such as an application server or an Internet server, or that includes a front-end component, such as a client computer having a graphical user interface or an Internet browser, or any combination thereof. The components of the system may be connected by any form or medium of digital data communication such as a communication network. Examples of communication networks include, e.g., a telephone network, a LAN, a WAN, and the computers and networks forming the Internet.

The computer system may include clients and servers. A client and server may generally be remote from each other and may typically interact through a network. The relationship of client and server may arise by virtue of computer programs running on the respective computers and having a client-server relationship to each other.

One or more features or steps of the disclosed embodiments may be implemented using an API. An API may define one or more parameters that are passed between a calling application and other software code (e.g., an operating system, library routine, function) that provides a service, that provides data, or that performs an operation or a computation.

The API may be implemented as one or more calls in program code that send or receive one or more parameters through a parameter list or other structure based on a call convention defined in an API specification document. A parameter may be a constant, a key, a data structure, an object, an object class, a variable, a data type, a pointer, an array, a list, or another call. API calls and parameters may be implemented in any programming language. The programming language may define the vocabulary and calling convention that a programmer will employ to access functions supporting the API.

In some implementations, an API call may report to an application the capabilities of a device running the application, such as input capability, output capability, processing capability, power capability, communications capability, etc.

While various embodiments have been described above, it should be understood that they have been presented by way of example and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments. For example, other steps may be provided, or steps may be eliminated, from the described flows, and other components may be added to, or removed from, the described systems. Accordingly, other implementations are within the scope of the following claims.

In addition, it should be understood that any figures which highlight the functionality and advantages are presented for example purposes only. The disclosed methodology and system are each sufficiently flexible and configurable such that they may be utilized in ways other than that shown.

Although the term “at least one” may often be used in the specification, claims and drawings, the terms “a”, “an”, “the”, “said”, etc. also signify “at least one” or “the at least one” in the specification, claims and drawings.

Finally, it is the applicant's intent that only claims that include the express language “means for” or “step for” be interpreted under 35 U.S.C. 112 (f). Claims that do not expressly include the phrase “means for” or “step for” are not to be interpreted under 35 U.S.C. 112 (f).

The presently described methods and systems are useful for detecting, predicting, treating and/or monitoring cancer status in a subject. Any appropriate subject, such as a mammal can be assessed, monitored, and/or treated as described herein. Examples of some mammals that can be assessed, monitored, and/or treated as described herein include, without limitation, humans, primates such as monkeys, dogs, cats, horses, cows, pigs, sheep, mice, and rats. For example, a human having, or suspected of having, cancer can be assessed using a method described herein and, optionally, can be treated with one or more cancer treatments as described herein.

A subject having, or suspected of having, any appropriate type of cancer can be monitored, assessed, and/or treated (e.g., by administering one or more cancer treatments to the subject) using the methods and systems described herein. A cancer can be any stage cancer. In some aspects, a cancer can be an early stage cancer. In some aspects, a cancer can be an asymptomatic cancer. In some aspects, a cancer can be a residual disease and/or a recurrence (e.g., after surgical resection and/or after cancer therapy). A cancer can be any type of cancer. Examples of types of cancers that can be assessed, monitored, and/or treated as described herein include, without limitation, lung, colorectal, prostate, breast, pancreas, bile duct, liver, CNS, stomach, esophagus, gastrointestinal stromal tumor (GIST), uterus and ovarian cancer. Additional types of cancers include, without limitation, myeloma, multiple myeloma, B-cell lymphoma, follicular lymphoma, lymphocytic leukemia, leukemia and myelogenous leukemia. In some aspects, the cancer is a solid tumor. In some aspects, the cancer is a sarcoma, carcinoma, or lymphoma. In some aspects, the cancer is lung, colorectal, prostate, breast, pancreas, bile duct, liver, CNS, stomach, esophagus, gastrointestinal stromal tumor (GIST), uterus or ovarian cancer. In some aspects, the cancer is a hematologic cancer. In some aspects, the cancer is myeloma, multiple myeloma, B-cell lymphoma, follicular lymphoma, lymphocytic leukemia, leukemia or myelogenous leukemia.

When treating a subject having, or suspected of having, cancer as described herein, the subject can be administered one or more cancer treatments. A cancer treatment can be any appropriate cancer treatment. One or more cancer treatments described herein can be administered to a subject at any appropriate frequency (e.g., once or multiple times over a period of time ranging from days to weeks). Examples of cancer treatments include, without limitation, surgical intervention, adjuvant chemotherapy, neoadjuvant chemotherapy, radiation therapy, hormone therapy, cytotoxic therapy, immunotherapy, adoptive T cell therapy (e.g., chimeric antigen receptors and/or T cells having wild-type or modified T cell receptors), targeted therapy such as administration of kinase inhibitors (e.g., kinase inhibitors that target a particular genetic lesion, such as a translocation or mutation), (e.g., a kinase inhibitor, an antibody, a bispecific antibody), signal transduction inhibitors, bispecific antibodies or antibody fragments (e.g., BiTEs), monoclonal antibodies, immune checkpoint inhibitors, surgery (e.g., surgical resection), or any combination of the above. In some aspects, a cancer treatment can reduce the severity of the cancer, reduce a symptom of the cancer, and/or to reduce the number of cancer cells present within the subject.

In some aspects, a cancer treatment can be a chemotherapeutic agent. Non-limiting examples of chemotherapeutic agents include: amsacrine, azacitidine, axathioprine, bevacizumab (or an antigen-binding fragment thereof), bleomycin, busulfan, carboplatin, capecitabine, chlorambucil, cisplatin, cyclophosphamide, cytarabine, dacarbazine, daunorubicin, docetaxel, doxifluridine, doxorubicin, epirubicin, erlotinib hydrochlorides, etoposide, fiudarabine, floxuridine, fludarabine, fluorouracil, gemcitabine, hydroxyurea, idarubicin, ifosfamide, irinotecan, lomustine, mechlorethamine, melphalan, mercaptopurine, methotrxate, mitomycin, mitoxantrone, oxaliplatin, paclitaxel, pemetrexed, procarbazine, all-trans retinoic acid, streptozocin, tafluposide, temozolomide, teniposide, tioguanine, topotecan, uramustine, valrubicin, vinblastine, vincristine, vindesine, vinorelbine, and combinations thereof. Additional examples of anti-cancer therapies are known in the art; see, e.g., the guidelines for therapy from the American Society of Clinical Oncology (ASCO), European Society for Medical Oncology (ESMO), or National Comprehensive Cancer Network (NCCN).

When monitoring a subject having, or suspected of having, cancer as described herein, the monitoring can be before, during, and/or after the course of a cancer treatment. Methods of monitoring provided herein can be used to determine the efficacy of one or more cancer treatments and/or to select a subject for increased monitoring.

In some aspects, the monitoring can include conventional techniques capable of monitoring one or more cancer treatments (e.g., the efficacy of one or more cancer treatments). In some aspects, a subject selected for increased monitoring can be administered a diagnostic test (e.g., any of the diagnostic tests disclosed herein) at an increased frequency compared to a subject that has not been selected for increased monitoring. For example, a subject selected for increased monitoring can be administered a diagnostic test at a frequency of twice daily, daily, bi-weekly, weekly, bi-monthly, monthly, quarterly, semi-annually, annually, or any at frequency therein.

In various aspects, DNA is present in a biological sample taken from a subject and used in the methodology of the invention. The biological sample can be virtually any type of biological sample that includes DNA. The biological sample is typically a fluid, such as whole blood or a portion thereof with circulating cfDNA. In embodiments, the sample includes DNA from a tumor or a liquid biopsy, such as, but not limited to amniotic fluid, aqueous humor, vitreous humor, blood, whole blood, fractionated blood, plasma, serum, breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, prostatic fluid, nipple aspirate fluid, lachrymal fluid, perspiration, check swabs, cell lysate, gastrointestinal fluid, biopsy tissue and urine or other biological fluid. In one aspect, the sample includes DNA from a circulating tumor cell.

As disclosed above, the biological sample can be a blood sample. The blood sample can be obtained using methods known in the art, such as finger prick or phlebotomy. Suitably, the blood sample is approximately 0.1 to 20 ml, or alternatively approximately 1 to 15 ml with the volume of blood being approximately 10 ml. Smaller amounts may also be used, as well as circulating free DNA in blood. Microsampling and sampling by needle biopsy, catheter, excretion or production of bodily fluids containing DNA are also potential biological sample sources.

The methods and systems of the disclosure utilize nucleic acid sequence information and can therefore include any method or sequencing device for performing nucleic acid sequencing including nucleic acid amplification, polymerase chain reaction (PCR), nanopore sequencing, 454 sequencing, insertion tagged sequencing. In some aspects, the methodology or systems of the disclosure utilize systems such as those provided by Illumina, Inc, (including but not limited to HiSeq™ X10, HiSeq™ 1000, HiSeq™ 2000, HiSeq™ 2500, Genome Analyzers™, MiSeq™. NextSeq, NovaSeq 6000 systems), Applied Biosystems Life Technologies (SOLID™ System, Ion PGM™ Sequencer, ion Proton™ Sequencer) or Genapsys or BGI MGI and other systems. Nucleic acid analysis can also be carried out by systems provided by Oxford Nanopore Technologies (GridiON™, MiniON™) or Pacific Biosciences (Pacbio™ RS II or Sequel I or II).

The present invention includes systems for performing steps of the disclosed methods and is described partly in terms of functional components and various processing steps. Such functional components and processing steps may be realized by any number of components, operations and techniques configured to perform the specified functions and achieve the various results. For example, the present invention may employ various biological samples, biomarkers, elements, materials, computers, data sources, storage systems and media, information gathering techniques and processes, data processing criteria, statistical analyses, regression analyses and the like, which may carry out a variety of functions.

Accordingly, the invention further provides a system for monitoring, detecting, analyzing, and/or assessing cancer. In various aspects, the system includes: (a) a sequencer configured to generate a low-coverage whole genome sequencing data set for a sample; and (b) a computer system and/or processor with functionality to perform a method of the invention.

In some aspects, the computer system further includes one or more additional modules. For example, the system may include one or more of an extraction and/or isolation unit operable to select suitable genetic components analysis, e.g., cfDNA fragments of a particular size.

In some aspects, the computer system further includes a visual display device. The visual display device may be operable to display a curve fit line, a reference curve fit line, and/or a comparison of both.

Methods for detection and analysis according to various aspects of the present invention may be implemented in any suitable manner, for example using a computer program operating on the computer system. As discussed herein, an exemplary system, according to various aspects of the present invention, may be implemented in conjunction with a computer system, for example a conventional computer system comprising a processor and a random access memory, such as a remotely-accessible application server, network server, personal computer or workstation. The computer system also suitably includes additional memory devices or information storage systems, such as a mass storage system and a user interface, for example a conventional monitor, keyboard and tracking device. The computer system may, however, include any suitable computer system and associated equipment and may be configured in any suitable manner. In one embodiment, the computer system comprises a stand-alone system. In another embodiment, the computer system is part of a network of computers including a server and a database.

The software required for receiving, processing, and analyzing information may be implemented in a single device or implemented in a plurality of devices. The software may be accessible via a network such that storage and processing of information takes place remotely with respect to users. The system according to various aspects of the present invention and its various elements provide functions and operations to facilitate detection and/or analysis, such as data gathering, processing, analysis, reporting and/or diagnosis. For example, in the present aspect, the computer system executes the computer program, which may receive, store, search, analyze, and report information relating to the human genome or region thereof. The computer program may comprise multiple modules performing various functions or operations, such as a processing module for processing raw data and generating supplemental data and an analysis module for analyzing raw data and supplemental data to generate quantitative assessments of a disease status model and/or diagnosis information.

The procedures performed by the system may comprise any suitable processes to facilitate analysis and/or cancer diagnosis. In one embodiment, the system is configured to establish a disease status model and/or determine disease status in a patient. Determining or identifying disease status may include generating any useful information regarding the condition of the patient relative to the disease, such as performing a diagnosis, providing information helpful to a diagnosis, assessing the stage or progress of a disease, identifying a condition that may indicate a susceptibility to the disease, identify whether further tests may be recommended, predicting and/or assessing the efficacy of one or more treatment programs, or otherwise assessing the disease status, likelihood of disease, or other health aspect of the patient.

The following example is provided to further illustrate the advantages and features of the present invention, but it is not intended to limit the scope of the invention. While this example is typical of those that might be used, other procedures, methodologies, or techniques known to those skilled in the art may alternatively be used.

### Example 1

### Detection of Cancer and Prediction of Overall Survival

In this example, the methodology of the present disclosure was utilized to detect cancer and predict overall patient survival. Exhibit A sets forth the study and results.

This study of prospectively enrolled individuals demonstrated the ability of the cfDNA fragmentation assay to distinguish between individuals with and without cancer. The assay of the invention displayed high performance in a multi-cancer setting using only fragmentation-related information obtained from low-coverage WGS.

The results suggest that machine learning models can differentiate between cancer and non-cancer despite the presence of common nonmalignant conditions (including cardiovascular, autoimmune, or inflammatory diseases) using cfDNA fragmentation profiles. Additionally, individuals with higher DELFI scores had a worse prognosis, independent of other characteristics.

These data support development of genome-wide cfDNA fragmentation analyses for noninvasive detection of both single and multiple cancers.

### Example 2

### Cell-Free DNA Fragmentation Profiling for Therapeutic Response Monitoring in Metastatic Cancer

The fraction of circulating tumor DNA (ctDNA) molecules in the plasma (ctDNA burden) has become a feasible measure to describe the overall tumor burden in patients with cancer. The ctDNA burden can change over time, lowering upon treatment response and rising as the tumor develops resistance to therapy. Monitoring the ctDNA dynamics throughout treatment can enable physicians to make timely treatment decisions. Ideally, this requires a fast, inexpensive, and generally applicable monitoring test that predicts therapeutic success and patient prognosis. Plasma ctDNA from liquid biopsies has great potential as a minimally invasive biomarker for tumor detection and response monitoring of (targeted) treatments. Plasma ctDNA is a dynamic tumor marker due to its short half-life and may detect relapse earlier than imaging and clinical parameters.

A variety of technologies exist for ctDNA profiling. Targeted next-generation sequencing (NGS) is a sensitive approach that can provide information about somatic abnormalities and detect a tumor's genomic changes. There are limitations to this approach, however, due to the prevalence of clonal hematopoietic variants within an aging population. A tissue or white blood cell-guided approach must be used to prevent these variants from obscuring the detection of tumor-specific alterations. On the other hand, single nucleotide variants can be tracked longitudinally using less expensive ctDNA hotspot mutation approaches, like droplet digital PCR (ddPCR). Because they detect a limited number of somatic tumor alterations, these hotspot mutation assays are not generally applicable to the diverse range of tumors within a patient population and provide a narrow view of a tumor's genetic makeup. For example, in patients with metastatic colorectal cancer (mCRC), a RAS/BRAF driver mutation that can be tracked is present in only half of the patients.

Prior research has shown that cell-free DNA (cfDNA) size distribution across the genome can be utilized to unravel its origin. The proportion of shorter fragments is larger in people with cancer than in healthy people. As survival of patients with cancer is inversely related to the stage of the disease, cfDNA fragment size compositions, i.e., the ratio of shorter versus longer cfDNA fragments, were exploited to develop a tool for early disease detection in patients with cancer. This approach, called DELFI (DNA evaluation of fragments for early interception), can distinguish cancer from non-cancers and indicate a tumor's origin. Due to the minimally invasive nature of the technology, cfDNA fragmentomics might also be of added clinical value for monitoring disease progression. Therefore, we developed the DELFI Tumor Fraction score (DELFI-TF), a machine-learning classifier capable of detecting tumor dynamics without needing genetic information about the tumor of origin. In the work, we evaluate the DELFI-TF classifier for treatment response monitoring in patients with mCRC.

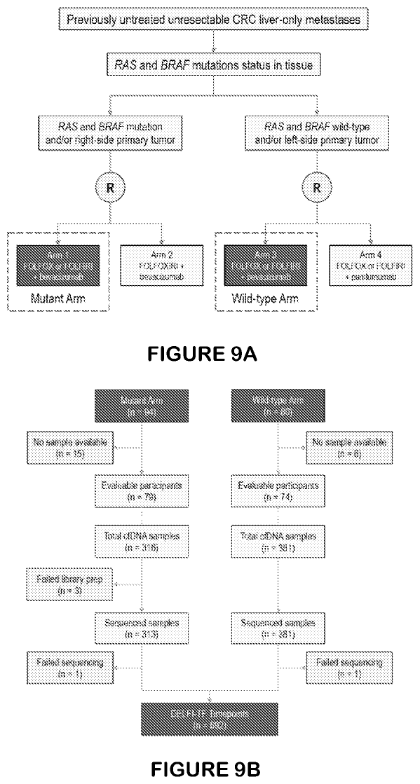

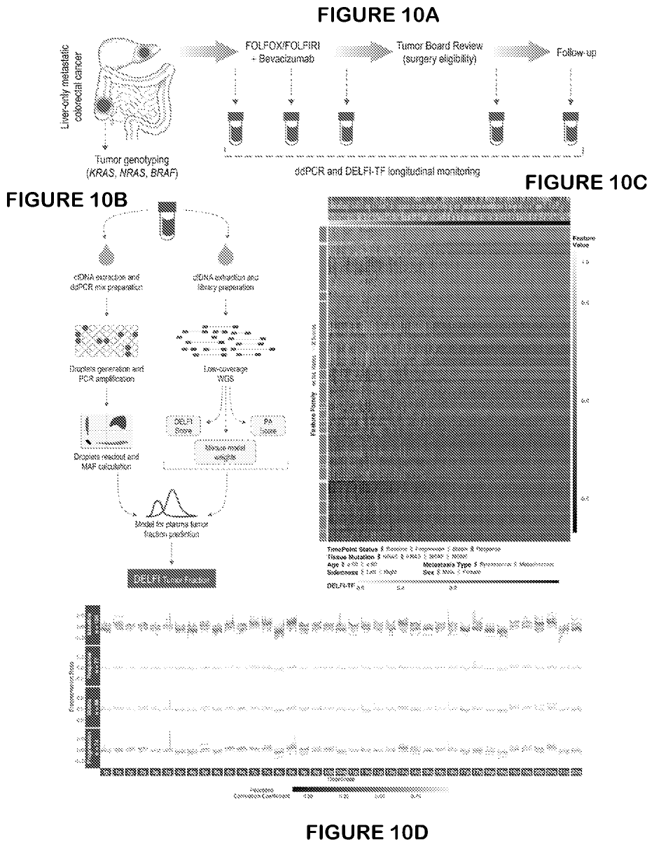

DELFI-TF model development using genome-wide cfDNA fragmentation profiles—692 serial plasma samples from patients with mCRC and RAS/BRAF-mutant (n=79) or RAS/BRAF-wild-type (n=74) disease participating in a prospective phase III clinical trial were processed and analyzed (CAIRO5) (Table 1, FIG. 9A and FIG. 9B). After study eligibility confirmation, including the unresectable status of liver metastases as defined by a central panel of experts, the mutation status of KRAS (exon 2, 3, and 4), NRAS (exon 2 and 3), and BRAF (codon 600) was assessed in available tissue samples, followed by blood draws at the pre-treatment baseline and at consecutive time points during treatment (FIG. 9A and FIG. 10). During an initial two-month period, patients in the mutant and wild-type arms were treated with a fluoropyrimidine-based first-line regimen (FOLFOX or FOLFIRI) plus bevacizumab (FIG. 9A and FIG. 10A). Hence, a radiological evaluation planned to assess liver metastases resectability was performed by a central tumor board review. Tumor-informed cfDNA analysis using droplet digital PCR (ddPCR) was retrospectively performed in 312 samples from patients in the mutant arm. Tumor-agnostic DELFI-TF analysis was successfully performed in 692 samples from patients in both mutant and wild-type arms (FIG. 9B and FIG. 10A). DELFI-TF failure rates associated with library preparation and whole-genome sequencing (WGS) were 0.42% and 0.29%, respectively (FIG. 9B). Participants were intended to be followed until death or study withdrawal.

TABLE 1
Demographics and clinical characteristics of study participants

| | CAIRO5 Arm 1 | | CAIRO5 Arm 3 | |
|---|---|---|---|---|
| | mutant | | wild-type | |
| | RAS/BRAF | | RAS/BRAF | |
| Characteristics | N = 79 | | N = 74 | |
| Age, years | | | | |
| Median (Range) | 62 | (41, 79) | 58.5 | (27, 76) |
| Sex | | | | |
| Male | 47 | (59%) | 47 | (64%) |
| Female | 32 | (41%) | 27 | (36%) |
| Mutation type | | | | |
| KRAS | 68 | (86%) | 0 | (0%) |
| NRAS | 5 | (6%) | 0 | (0%) |
| BRAF | 6 | (8%) | 2 | (3%) |
| None | 0 | (0%) | 72 | (97%) |
| Primary tumor sidedness | | | | |
| Right | 27 | (34%) | 2 | (3%) |
| Left | 52 | (66%) | 72 | (97%) |
| Type of liver metastasis | | | | |
| Metachronous | 9 | (11%) | 10 | (14%) |
| Synchronous | 70 | (89%) | 64 | (86%) |
| Surgical complete resection | | | | |
| No | 53 | (67%) | 38 | (51%) |
| Yes | 26 | (33%) | 36 | (49%) |
| ECOG performance status | | | | |
| 0 | 46 | (59%) | 46 | (62%) |
| 1 | 32 | (41%) | 28 | (38%) |
| Unknown | 1 | (1.2%) | 0 | (0%) |
| Progression-free survival status | | | | |
| No Progression or Death | 5 | (6.3%) | 14 | (19%) |
| Progression or Death | 74 | (94%) | 60 | (81%) |
| Median (Range) PFS, months | 9.4 | (1.9, 58.9) | 11 | (2.1, 68.4) |
| Overall survival status | | | | |
| Alive | 21 | (27%) | 42 | (57%) |
| Death From Any Cause | 58 | (73%) | 32 | (43%) |
| Median (Range) OS, months | 23.4 | (3.6, 58.9) | 28.5 | (3.5, 68.4) |

### OS, Overall Survival; PFS, Progression-Free Survival

To perform a mutation-independent assessment of cancer-specific alterations in cfDNA, the DELFI-TF model was first designed (FIG. 10B). For all cfDNA samples of patients in the mutant arm (n=312), the tumor burden was initially quantified as the mutant allele frequency (MAF) of the tumor-tissue-proven RAS/BRAF variant measured by ddPCR. Using duplicate aliquots of cfDNA samples, genome-wide fragment-sequencing statistics were obtained through low-coverage WGS of the cfDNA libraries (FIG. 10B). A Bayesian hierarchical regression model was trained and cross-validated against the MAF of the tumor-specific driver RAS/BRAF variant measured by ddPCR in all longitudinal cfDNA samples sequenced in the mutant arm. To generate a predicted DELFI-TF value for each sample, this model considered the DELFI scores, the plasma aneuploidy (PA) scores, and the weight components from a mixture model that utilizes cfDNA fragment size densities (FIG. 10B). We performed an unsupervised clustering analysis using short-to-long ratios of fragments sizes across 504 5-Mb bins and arm-level copy number z-scores for 39 chromosomal arms across baseline and on-treatment time points of 128 patients with mCRC (FIG. 10C). Remarkably, the fragmentation profile differences could be observed in multiple regions throughout the genome for the vast majority of patients with mCRC at the baseline time point across several clinical and demographic characteristics, mostly corresponding to high DELFI-TF values. Similarly, we observed that the majority of time points associated with progressive disease by imaging assessment also presented marked heterogeneity and high tumor fractions, contrasting with the majority of time points associated with stable disease or radiologic response after the start of first-line systemic therapy, which was associated with fewer genomic abnormalities and DELFI-TF values (FIG. 10C and FIG. 10D). These findings suggest that our model designed to predict tumor fraction in cfDNA is capable of real-time identification of systemic treatment response in a non-invasive manner.

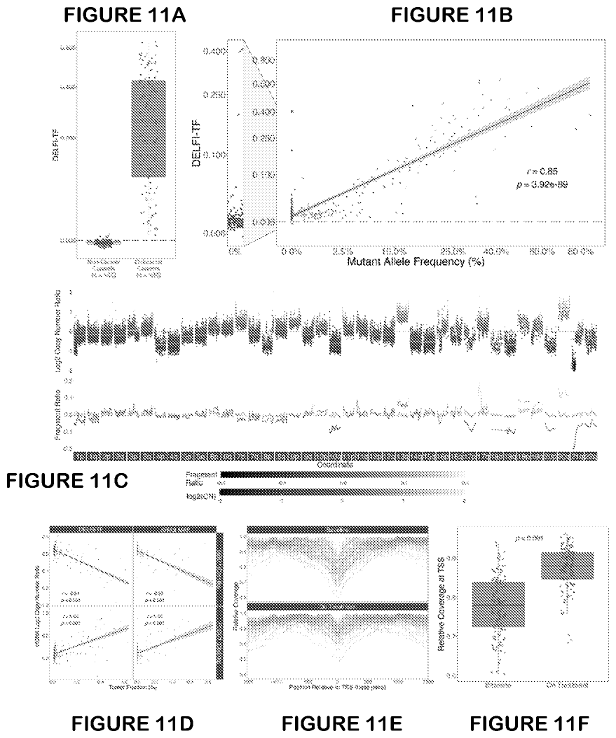

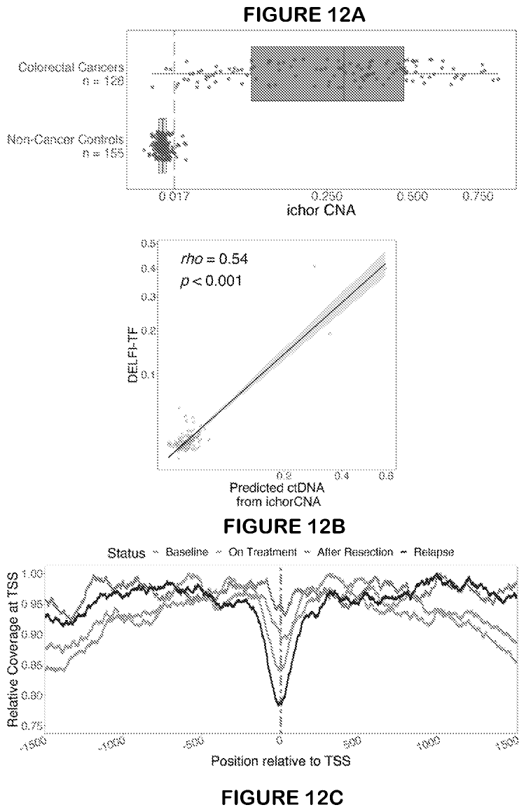

DELFI-TF accurately reflects cfDNA mutant allele frequencies and copy number changes—An independent analysis of the DELFI-TF model was performed using non-cancer control samples (n=155) from a Danish cohort of symptomatic patients with a prior negative work-up for cancer diagnosis. Compared with treatment-naïve samples (n=128) from the CAIRO5 cohort, non-cancer control samples exhibited significantly lower DELFI-TF values, with a 95% confidence interval (CI) upper limit of 0.006. Notably, all treatment-naïve samples from patients with mCRC had DELFI-TF values significantly higher than 0.006 (FIG. 11A). We observed similar distributions when samples from these two cohorts were assessed with ichorCNA, a tool designed to estimate tumor fractions in ultra-low pass WGS data (FIG. 12A). However, the non-cancer controls exhibited a slightly wider range of tumor fraction values, including a higher 95% CI upper limit (0.017). In addition, the ichorCNA values for a few CRC samples overlapped with estimated tumor fractions for the non-cancer controls, suggesting that DELFI-TF more precisely reflects the disease burden detected in cfDNA samples.

Next, the analytical performance of the DELFI-TF in comparison to the mutation-based tumor burden assessment was evaluated using ddPCR for RAS/BRAF MAF quantification (FIG. 11B). A strong correlation was observed between MAF and DELFI-TF values (Pearson r=0.85, p=3.92e-89). Interestingly, a wide range of positive DELFI-TF values among 60 (20.5%) on-treatment time points with undetectable RAS/BRAF MAF by ddPCR was observed, implying that the DELFI-TF approach may be more sensitive for measuring tumor fractions in patients receiving effective antineoplastic treatments. It further confirmed that these discordant time points with undetectable RAS/BRAF MAF presented DELFI-TF values that correlated with tumor fractions predicted by ichorCNA (Spearman rho=0.54, p<0.001) (FIG. 12B). The association between cfDNA fragmentomes and copy number changes in tissue (FIG. 11C) and plasma samples (FIG. 11D) were examined. Patient-matched formalin-fixed paraffin-embedded (FFPE) tumor tissue was analyzed via low-pass WGS (average coverage 0.2×) for 104/153 patients. We consistently observed that abnormal cfDNA fragmentation profiles were present in regions of the genome that were copy-neutral and were further affected in regions with copy-number changes in tissue samples (FIG. 11C). In addition, we showed equivalent analytical performance and significant correlations between tumor fractions assessed by DELFI-TF and ddPCR MAF, in relation to cfDNA copy number for MBD1 (Pearson, DELFI-TF r=0.64, p<0.001, ddPCR MAF r=−0.67, p<0.001) and PLGC1 (Pearson, DELFI-TF r=−0.84, p<0.001; ddPCR MAF r=0.55, p<0.001), genes commonly deleted and amplified in mCRC, respectively (FIG. 11D). The ability to detect nucleosome depleted regions using relative coverage at the transcription start sites (TSS) as a surrogate marker for gene expression in CRC was explored. It was observed that the relative coverage at the TSS for a group of approximately 900 genes highly expressed in CRC was significantly lower for samples at baseline than for samples on treatment (Wilcoxon, p<0.001) (FIG. 11E and FIG. 11F), reflecting dynamic changes detected during disease response and progression (FIG. 12C). Altogether, DELFI-TF accurately captures cancer-specific alterations related to MAFs and copy number aberrations in cfDNA.

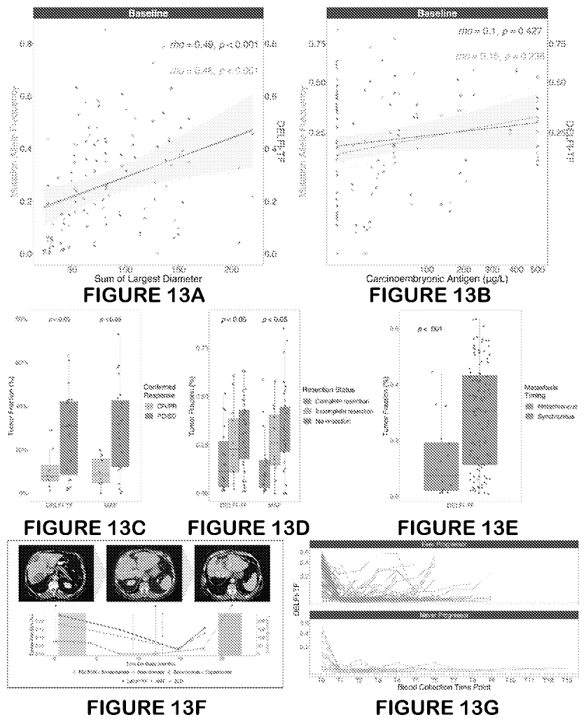

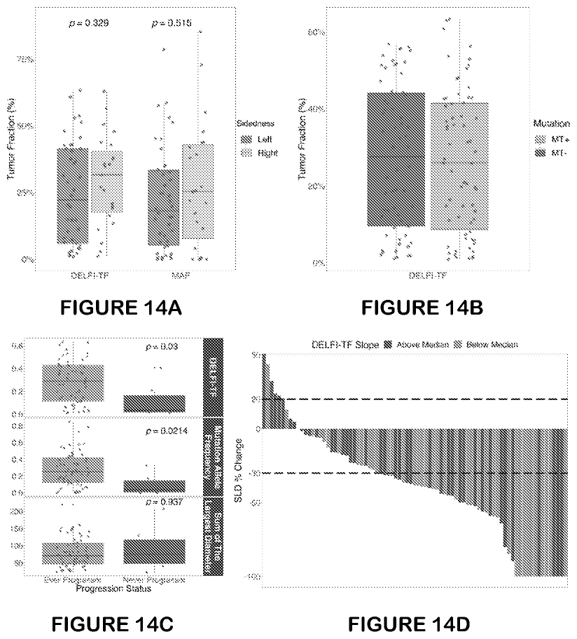

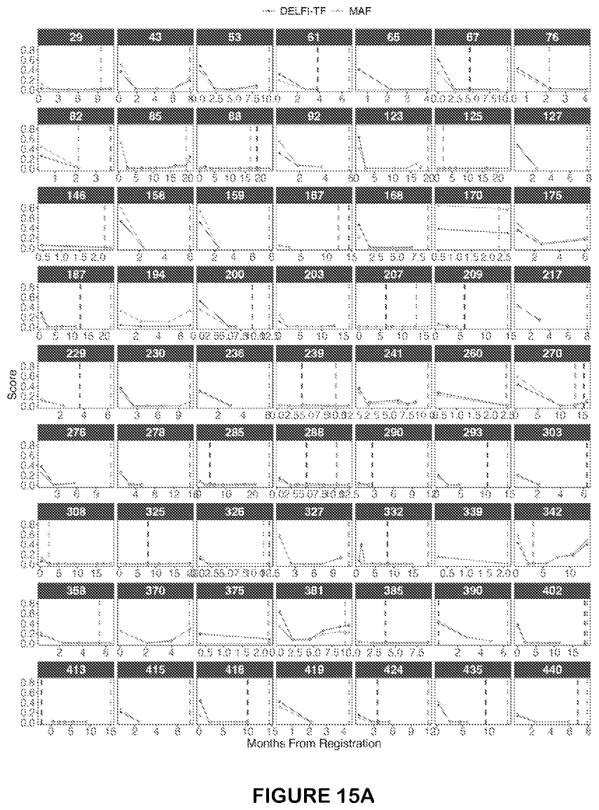

DELFI-TF correlates with clinical features and standard imaging assessment—The DELFI-TF approach with clinical patient characteristics were compared. At the treatment-naïve time point, a modest correlation between DELFI-TF and ddPCR MAF with the sum of the longest diameters (SLD) of the target metastatic lesions in the liver was observed (DELFI-TF Spearman rho=0.49, p<0.001; ddPCR MAF Spearman rho=0.48, p<0.001) (FIG. 13A). In contrast, no correlation was seen with serum carcinoembryonic antigen (CEA) levels measured before treatment initiation (DELFI-TF Spearman rho=0.1, p=0.43; ddPCR MAF Spearman rho=0.15, p=0.24) (FIG. 13B). Baseline DELFI-TF and ddPCR MAF tumor fractions did correlate with clinical response, as pre-treatment levels were significantly lower in patients with a later-confirmed partial or complete response than in patients with stable or progressive disease by two consecutive scans (Wilcoxon, DELFI-TF p<0.05; ddPCR MAF p<0.05) (FIG. 13C). Furthermore, patients that were deemed resectable after systemic induction therapy had significantly lower baseline tumor fractions (Kruskal-Wallis, DELFI-TF p<0.05; ddPCR MAF p<0.05) (FIG. 13D), as did patients with metachronous disease (Wilcoxon, p<0.05) (FIG. 13E). Of note, DELFI-TF and ddPCR MAF tumor fractions did not differ by tumor sidedness (FIG. 14A) or RAS/BRAF mutation status (FIG. 14B). At baseline, DELFI-TF and ddPCR MAF tumor fractions were significantly lower in patients who never had disease progression at any time point during the CAIRO5 trial (Never Progressors) than in patients who experienced progressive disease at some point during treatment (Ever Progressors) (FIG. 14C). On the other hand, an analysis using the SLD of target liver lesions at baseline could not distinguish Never from Ever Progressors (FIG. 14C). Moreover, it was demonstrated that DELFI-TF could accurately track longitudinal disease burden dynamics, even at late time points in patients treated with curative-intent liver metastases resection (FIG. 13C). Overall, it was confirmed that Ever Progressors more often exhibited increasing DELFI-TF values at early time points and emerging disease progression at late time points than Never Progressors (FIG. 13G).

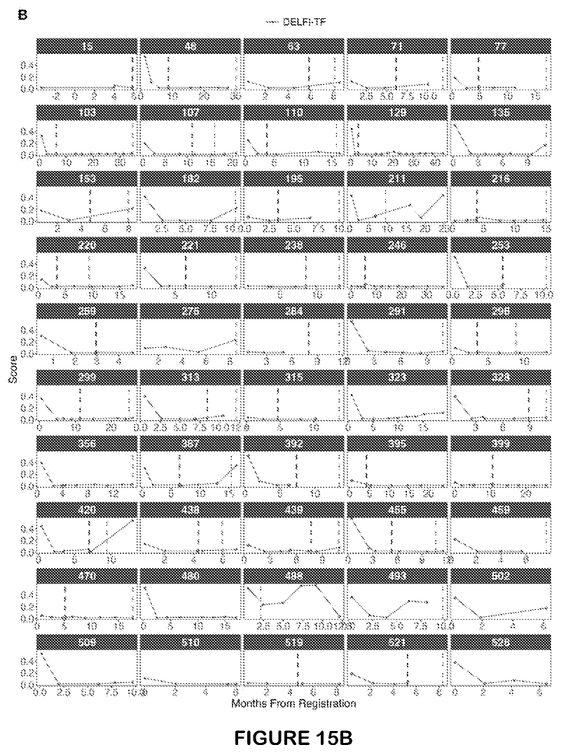

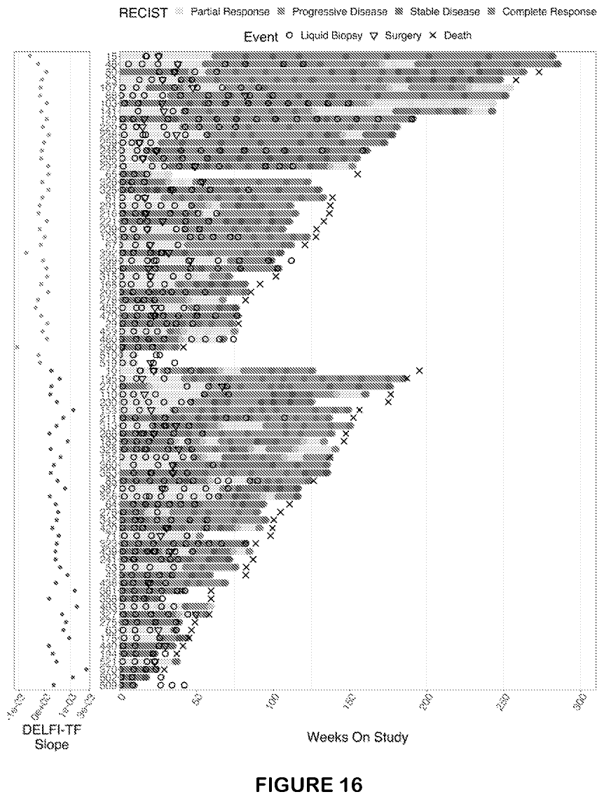

Once irt was verified that the analytical equivalence between the DELFI-TF model and the ddPCR assay for RAS/BRAF MAF assessment, it was decided to decided to further explore the association of dynamic changes of DELFI-TF and clinical outcomes. In order to accommodate the longitudinal evolution of consecutive DELFI-TF values in a single score, the DELFI-TF slope was calculated, which is defined as the slope of the line fitted to the DELFI-TF values using linear-regression, starting at the first blood biopsy time point after treatment initiation and ending at the time of disease progression confirmed by RECIST1.1. It was then observed a trend towards lower DELFI-TF slopes for patients who experienced a partial or complete response, as their best overall response (Fisher exact test, p=0.1) (FIG. 14E). Overall, the temporal analysis of DELFI-TF and ddPCR MAF showed comparable tumor dynamics (FIG. 15A). For patients in the wild-type arm, the temporal analysis could only be performed using the DELFI-TF values (FIG. 15B). Patients with DELFI-TF slopes below the median had higher rates of objective radiologic responses to the first-line treatment and longer durations of follow-up than patients with DELFI-TF slopes above the median (FIG. 16).

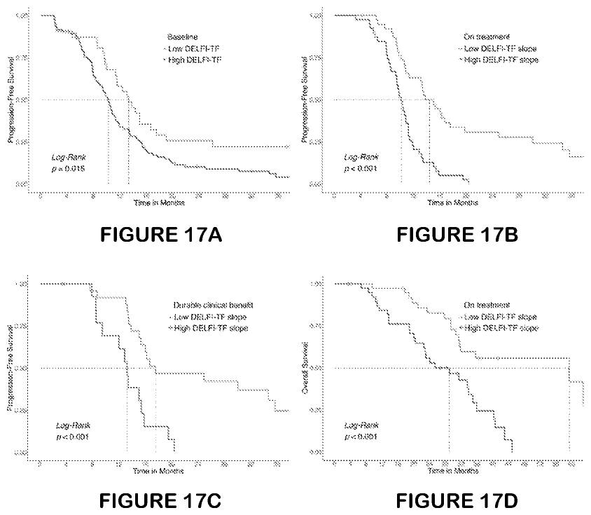

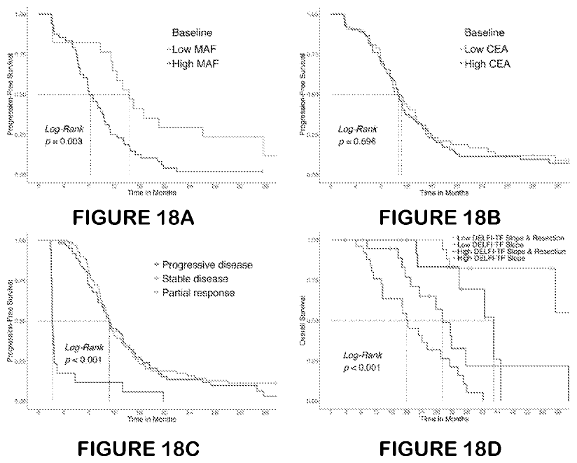

Subsequently, the baseline DELFI-TF and DELFI-TF slopes were correlated with survival outcomes. At baseline, patients with DELFI-TF values lower than first quartile showed longer median progression-free survival (PFS) than patients with DELFI-TF above the first quartile (13.4 months vs 10.2 months, hazard ratio [HR]=1.77, 95% CI 1.12 to 2.78, Log-rank p=0.013) (FIG. 17A). For the RAS/BRAF mutant arm, the baseline MAF for tumor fraction assessment showed similar distinction in median PFS (14.4 months vs 8.3 months, HR=2.56, 95% CI 1.36 to 4.83, Log-rank p=0.00272) (FIG. 18A). Serum CEA levels at baseline were unable to predict disease progression or death (FIG. 18B). Within the trial, patients were evaluated two months after the start of therapy by an expert panel to assess the resectability of the liver metastases. This first clinical response evaluation could not distinguish survival differences between patients with partial response and stable disease (11.3 months vs 11.2 months, HR=1.13, 95% CI 0.79 to 1.61, Log-rank p=0.52) (FIG. 18C). Patients with DELFI-TF slope below the median presented with longer PFS in the overall study population (13.4 months vs 10.4 months, HR=2.03, 95% CI 1.247 to 3.318, Log-rank p=3.76e-3) (FIG. 17B) and in patients who experienced durable clinical benefit, defined as an objective response or stable disease longer than 12 months (16.7 months vs 13.3 months, HR=2.235, 95% CI 1.097 to 4.553, Log-rank p=0.023) (FIG. 17C). Patients with below-the-median DELFI-TF slopes also experienced significantly longer overall survival (OS; 59.4 months vs 29.1 months, HR=3.05, 95% CI 1.58-5.90, Log-rank p=5.135c-4) (FIG. 17D). We also observed that survival could be further stratified by resection status, with a better OS outcome for patients with complete resection of the primary tumor and liver metastases than for patients with incomplete resection or none (FIG. 17D).

Liquid biopsies cfDNA analyses are a new and promising clinical tool in cancer research. A DELFI-TF score was developed, a fragmentomics approach able to measure tumor burden quantitatively, and showed its potential for longitudinal disease monitoring in patients with mCRC.

Currently, liquid biopsy ctDNA testing for the presence of cancer mostly depends on the detection of one or more somatic tumor alterations. Different research advantages have utilized the cfDNA fragmentomics trait as an alternative feature. In vitro and in silico size selection of cfDNA molecules, i.e., selecting for shorter over longer cfDNA fragments, can enrich ctDNA and enhance the identification of genetic alterations in ctDNA. Alternatively, genome-wide fragmentation profiles can facilitate tumor detection and identification of the tumor of origin. The novelty of our cfDNA fragmentomics approach is the possibility to longitudinally monitor patient response using low-coverage whole-genome sequencing of minute amounts of cfDNA, without a requirement for detecting driver mutations.

Despite diagnostic and treatment advances, most patients with mCRC relapse, providing the clinical need for a biomarker to guide the treatment course. Yet, currently available follow-up methods like clinical imaging and serum CEA have limited accuracy for detecting the viability of tumor tissue and assessing treatment effectiveness shortly after the start of therapy is therefore challenging. The current study showed that DELFI-TF might be more sensitive than conventional approaches for treatment response monitoring as DELFI-TF could predict PFS better than serum CEA measurements and clinical computed tomography (CT) imaging after treatment initiation. Identifying treatment response or progression provides physicians with the opportunity to adapt a patient's' treatment regimen.

Aside from DELFI-TF, the ddPCR MAF after treatment initiation was also prognostic for disease recurrence. However, the ability to detect differences in PFS among patients with undetectable ddPCR MAF suggests that DELFI-TF may be more sensitive for treatment response monitoring, although a fragmentomics monitoring approach cannot track treatment-induced genomic changes in the tumor, which is possible with targeted sequencing approaches. Furthermore, both the ddPCR MAF and the DELFI-TF prior to treatment were indicative for the success rate of complete resection of the liver metastases and OS. The DELFI-TF, however, has conceptual advantages over hotspot mutation assays like ddPCR. Since the DELFI-TF does not require prior knowledge of the tumor's driver alterations, it is generally applicable to samples from patients with any cancer type. The low-coverage WGS needed for the fragmentation profile is less costly than targeted sequencing. As the tumor burden can fluctuate over time, lowering upon treatment response and rising as the tumor develops resistance to therapy, the DELFI-TF can be utilized as a tool to highlight the right moment for a more elaborate panel sequencing analysis.

Within the limited number of patients with blood samples after liver resection, a positive DELFI-TF post-operatively seemed to indicate disease recurrence with modest sensitivity. Yet, the blood tests close to surgery might have been a confounding factor in the training cohort. Measurements within 48 hours after surgery showed spikes in the DELFI-TF. Since surgery is an invasive procedure, samples taken too close in time to surgery may represent wound healing rather than tumor-derived cfDNA. Therefore, the cut-off for positive DELFI-TF results in samples taken after complete resection, i.e., the minimal residual disease setting, should be further investigated.

Here, the DELFI-TF was assessed and applied orthogonal validation on a sample level to a single-nucleotide variant genotyping approach using samples derived from patients with mCRC collected in a well-controlled clinical trial. Thereby, the DELFI-TF was defined and its potential prognostic power to detect disease progression over conventional approaches for treatment response monitoring was shown in the training set. We caution that these results must be confirmed in the validation cohort and afterward also evaluated for other types of cancer or earlier stages of disease before clinically applicable. These results are not directly transferable to other bodily fluids like urine and cerebrospinal fluid as they have different distributions of cfDNA fragments. In conclusion, we developed a novel quantitative measure of ctDNA burden using cfDNA fragmentomics. Within the training cohort, the DELFI-TF appears to be a useful non-invasive approach to monitor therapeutic success in patients with mCRC.

Study design and population—The present study is a retrospective analysis of liquid biopsies collected from a homogenous group of patients with mCRC participating in the prospective CAIRO5 clinical trial (NCT02162563). The phase III randomized CAIRO5 trial investigates the optimal first-line systemic therapy for patients with histologically proven CRC with isolated, previously untreated, initially unresectable liver metastases. Patients treated with doublet chemotherapy (FOLFOX or FOLFIRI) and bevacizumab with at least one blood draw prior to and after treatment were included in the present study. All patients were considered unresectable at inclusion, i.e., R0-resection could not be achieved in one procedure with one surgical intervention. Upon treatment with doublet chemotherapy and bevacizumab, patients were evaluated every two months by an expert panel of liver surgeons and abdominal radiologists for the possibility of local treatment of colorectal liver metastases following current clinical practice. Clinical follow-up was performed according to the standard of care, including a clinical review every three months and CT imaging and serum CEA every six months. When the liver metastases stayed unresectable, chemotherapy was continued without the targeted agent for the total duration of pre- and post-operative treatment of six months, and patients were continuously evaluated until the progression of the disease by serum CEA and CT imaging every two months. Follow-up was recorded until Sep. 1, 2021. The trial was approved by a medical ethical committee, performed according to the Declaration of Helsinki, and patients signed written informed consent for study participation and blood collection for translational research.

Blood collection and cfDNA extraction-Collection of liquid biopsy samples was performed at the medical center of inclusion prior to study treatment (baseline), pre-operatively, post-operatively and every three months during follow-up until disease progression or treatment completion. Blood samples were taken using 10 mL cell-free DNA BCT® tubes (Streck, La Vista, USA) and collected centrally at the Netherlands Cancer Institute (Amsterdam, the Netherlands). A two-step centrifugation process, 10 minutes at 1700×g and 10 minutes at 20 000×g, isolated the cell-free plasma. The cell-free plasma was stored at −80° C. until further use. Isolation of cfDNA was performed using the QIAsymphony (Qiagen, Germany) with an elution volume of 60 μL. The cfDNA concentration was assessed using the Qubit™ dsDNA High-Sensitivity Assay (ThermoFisher; Waltham, MA, USA). As input for the library preparation, aliquots of a maximum of 15 ng were made and added up to 51 μL using TE buffer when necessary. The cfDNA aliquots were shipped to the laboratory at Delfi Diagnostics (Baltimore, MD, USA).

Library preparation and cfDNA sequencing-Upon arrival at the laboratory, the extracted cfDNA was qualified using the TapeStation 4200 (Agilent Technologies; Santa Clara, CA, USA). NGS libraries were constructed using the NEBNext DNA Library Prep kit (New England Biolabs; Ipswich, MA, USA) with up to 15 ng of cfDNA input, as previously described (19), with four main modifications to the manufacturer's guidelines: 1) the library purification steps used the on-bead AMPure XP (Beckman Coulter; Brea, CA, USA) approach to minimize sample loss during elution and tube transfer steps, 2) NEBNext End Repair, dA-tailing, and adapter ligation enzyme and buffer volumes were adjusted as appropriate to accommodate the on-bead AMPure XP strategy, 3) Illumina dual-index adapters were utilized in the ligation reaction, and 4) cfDNA libraries were amplified for four cycles with Phusion HotStart Polymerase (ThermoFisher; Waltham, MA, USA). WGS library quality was determined using the 2100 Bioanalyzer (Agilent Technologies; Santa Clara, CA, USA) or the TapeStation 4200 (Agilent Technologies; Santa Clara, CA, USA). Next, a total of 96 dual-indexed cfDNA libraries containing samples with distinct barcodes were pooled together into a single lane of an S4 flow cell, and 100-bp paired-end (200 cycles) WGS sequencing was performed on the NovaSeq 6000 (Illumina; San Diego, CA, USA), aiming 8× coverage per genome. To limit batch effects, all samples collected from the same individual had libraries created in the same batch, including a duplicate library as an inter-batch control and a technical replicate of nucleosomal DNA obtained from nuclease-digested human peripheral blood mononuclear cells as an intra-batch control. RAS/BRAF mutation analyses-RAS and BRAF V600E mutation analyses were performed on tumor tissue DNA following routine clinical practice for all patients. For the subset of patients with a RAS/BRAF tumor tissue mutation, longitudinal liquid biopsy hotspot mutation analyses by ddPCR (Bio-Rad, Hercules, CA, USA) and fragmentation analyses were performed. The ddPCR™ KRAS G12/G13 (#1863506), ddPCR™ KRAS Q61 (#12001626), ddPCR™ KRAS A146T (#10049550), and the ddPCR™ BRAF V600 (#12001037) Screening Kits were used according to the manufacturer's instruction, using 9 μL of sample, 11 μL of ddPCR supermix for probes (no dUTP), 1 μL of the multiplex assay and 1 μL of nuclease-free water. All measurements were performed in duplicate, including a blank (nuclease-free water) and a positive control. Patients with a RAS/BRAF mutation that could not be tracked by ddPCR were excluded (FIG. 9B). Data were analyzed using the QuantaSoft™ software version 1.6.6 (Bio-Rad, Hercules, CA, USA) and an automated correction algorithm as previously described.

Analyses of cfDNA sequencing data-On a per-sample basis, the paired-end sequenced reads were aligned to a reference genome (hg19) using paired-end alignment with Bowtie (version 2.3.0). The aligned reads were sorted and converted to BAM and subsequently to BED format using Samtools (version 1.3.1) and Bedtools (version 2.26.0), respectively. Fragment lengths were calculated based on start and end coordinates, and the fragments were divided into 504 5-Mb bins, covering approximately 2.6 Gb of the genome. Next, the number of short (100-150 bp) and long (151-220 bp) fragments per bin was calculated using R/Bioconductor (version 3.6.2), and these counts were corrected by GC content as described by Benjamini and Speed. The corrected count of short fragments was divided by the corrected count of long fragments by bin to obtain the fragmentation profile per person.

Four statistics were calculated for each sample to generate the DELFI-TF score DELFI score, DELFI divergence, mixture model components, and arm-level aneuploidy scores. The DELFI score was calculated similarly to the method described by Cristiano et al. (Genome-wide cell-free DNA fragmentation in patients with cancer. Nature 570, 385-389 (2019)), and indicates how similar the fragmentation profile looks to an individual with cancer or an individual without cancer. The DELFI divergence is defined as one minus the correlation between the binned-and-mean-centered short-to-long ratios of a given sample and those of the “median healthy” sample from a reference cohort containing only healthy samples. The mixture model summarizes the fragment-size distributions, and the weight statistics from this model are evaluated when generating the DELFI-TF.

Using these statistics calculated per sample, a Bayesian hierarchical regression model was trained against the allele frequencies of the tumor-specific driver RAS/BRAF variant measured by ddPCR in the longitudinal cfDNA samples using R. This model takes the DELFI Score, DELFI divergence, mixture model weights, and an cuploidy score as inputs and outputs a predicted MAF. The model assumes MAF is a beta-distributed random variable and assumes that the expected MAF of a given sample is functionally related to the described features via the inverse-logit of the feature-matrix multiplied by a vector of regression coefficients plus a patient-specific random intercept that accounts for the within-patient correlation between measurements. To generate unbiased predictions, avoid overfitting, and assess generalizability, training is done via leave-one-patient-out cross-validation. In this cross-validation scheme, each patient's data is held out in turn, the model is trained on the remaining samples, and that trained model is then used to generate predictions for the held-out samples. DELFI-TF was defined as the predicted MAF from this cross-validation scheme. We evaluate the quality of the generated predictions by assessing the correlation of these predictions with the observed ddPCR MAF values and by evaluating the relationship between those predictions and time to progression or death. DELFI-TF dynamics analysis—To capture the molecular dynamics of tumor burden over time, we computed DELFI-TF slope, that is, the slope of the regression line fitted to the DELFI-TF values at time Tl onward until before the progression for the PFS analysis and up to 60 days after the progression date for the OS analysis. For this practice we selected the patients that had at least 3 collected samples before the progression, and at least one of those samples was collected in the progression window, which was 120 days before until the progression date for PFS analysis (79 patients) and 120 days before until 60 days after the progression for the OS analysis (80 patients). The regression lines are computed using Python/scikit-learn (version 3.9.13/1.1.1).

Relative coverage computation for gene expression analysis—For this analysis we selected a set of 854 transcripts identified from the Broad GDAC Firehose Pipeline that are known to be highly expressed in colon adenocarcinoma and extracted their transcription starting site (TSS) coordinates. The fragment coverage was calculated at these TSSs plus a flanking region of 1,500 bp on each side for all genes on only the 126 patients who had plasma samples at both TO and Tl timepoints. The list of TSS coordinates and the aligned fragments were in the BED format and the coverage calculation was performed using pybedtools (version 0.9.0), a python interface of Bedtools.

Statistical analyses—Correlations between DELFI-TF and ddPCR MAF were calculated using Pearson's correlation coefficient. Similarly, correlations between DELFI-TF/ddPCR MAF and copy number ratios were calculated using Pearson's correlation. Spearman correlation tests were used between DELFI-TF/ddPCR MAF and the SLDs and serum CEA. All two-sample hypothesis testing excluding survival analysis were performed using a Wilcoxon rank sum test. The tumor fraction based on DELFI-TF and MAF between resection status was compared using a Kruskal-Wallis test. Survival analyses were performed using Mantel-Cox log-rank tests. Analyses were performed with R Statistical Software (version 4.2.1 Foundation for Statistical Computing, Vienna, Austria). Unless otherwise noted, hypothesis tests were two-sided with a type 1 error of 5% for determining statistical significance.

Although the invention has been described with reference to the presently preferred embodiments, it should be understood that various modifications can be made without departing from the spirit of the invention. Accordingly, the invention is limited only by the following claims.

What is claimed is:

1. A method of monitoring cancer comprising:
determining a cell-free DNA (cfDNA) fragmentation profile of a sample from a subject;
calculating a fragmentation score based on the cfDNA fragmentation profile, the score being indicative of a likelihood of presence of cancer in the subject;
determining a ratio of short to long fragments and a fragment size distribution from the fragmentation profile;
calculating a divergence score based on the ratio of short to long fragments in the sample as correlated to a ratio from a sample from a healthy subject;
determining a set of model weights based on a fragment size distribution;
training a machine learning model using a set of features extracted from a plurality of fragmentation profiles of multiple subjects; and
determining, by the machine learning model, a monitoring score for the sample based on the fragmentation score, the divergence score, and the model weights, the monitoring score being indicative of a level of a tumor-derived nucleic acid in the cfDNA of the sample.

2. The method of claim 1, wherein the divergence score is indicative of a correlation between the fragmentation profile and a median fragmentation profile observed for healthy subjects.

3. The method of claim 1, wherein the monitoring score has a range of 0 to 1.

4. The method of claim 1, further comprising determining at least one of a likelihood of overall survival, a likelihood of progression free survival, or a time to progression of the subject based on the monitoring score.

5. The method of claim 4, wherein the likelihood of overall survival, the likelihood of progression free survival, or the time to progression of the subject decrease with an increase in monitoring score value.

6. The method of claim 5, further comprising classifying the monitoring score as a high score or a low score, wherein a high score is indicative of at least one of decreased overall survival, decreased progression free survival, or decreased time to progression of the subject.

7. The method of claim 6, wherein the monitoring score is classified before a cancer treatment is administered to the subject to provide a baseline classification for the subject.

8. The method of claim 6, wherein the monitoring score is classified at a first time point after a cancer treatment is administered to the subject to provide a post treatment classification for the subject.

9. The method of claim 1, wherein the likelihood of cancer progression in the subject increases with an increase in monitoring score value.

10. The method of claim 1, wherein the likelihood of cancer progression in the subject decreases with a decrease in monitoring score value.

11. The method of claim 1, wherein the likelihood of a positive response to a cancer treatment administered to the subject increases with a decrease in monitoring score value.

12. The method of claim 1, wherein the likelihood of a positive response to a cancer treatment administered to the subject decreases with an increase in monitoring score value.

13. The method of claim 1, wherein the monitoring score predicts a fraction of tumor-derived nucleic acid or a clonal mutant allele fraction of a mutation associated with a cancer.

14. The method of claim 1, wherein the cancer is a solid tumor.

15. The method of claim 1, wherein the cancer is a sarcoma, carcinoma, or lymphoma.

16. The method of claim 1, wherein the cancer is selected from the group consisting of: colorectal, lung, kidney, brain, prostate, breast, pancreas, bile duct, liver, CNS, stomach, esophagus, gastrointestinal stromal tumor (GIST), uterus and ovarian cancer.

17. The method of claim 1, wherein the cancer is a hematologic cancer.

18. The method of claim 1, wherein the cancer is selected from the group consisting of: myeloma, multiple myeloma, B-cell lymphoma, follicular lymphoma, lymphocytic leukemia, leukemia and myelogenous leukemia.

19. The method of claim 1, further comprising administering a cancer treatment to the subject.

20. The method of claim 17, wherein the cancer treatment is selected from the group consisting of surgery, adjuvant chemotherapy, neoadjuvant chemotherapy, radiation therapy, hormone therapy, cytotoxic therapy, immunotherapy, adoptive T cell therapy, targeted therapy, or any combination thereof.

21. The method of claim 1, wherein the cfDNA fragmentation profile is determined by:
obtaining and isolating cfDNA fragments from the subject;
sequencing the cfDNA fragments to obtain sequenced fragments;
mapping the sequenced fragments to a genome to obtain windows of mapped sequences; and
analyzing the windows of mapped sequences to determine cfDNA fragment lengths and generate the cfDNA fragmentation profile.

22. A method of determining at least one of overall survival, progression free survival, or time to progression comprising:
determining a cell-free DNA (cfDNA) fragmentation profile of a sample from a subject;
calculating a fragmentation score based on the cfDNA fragmentation profile, the score being indicative of a likelihood of presence of cancer in the subject;
determining a ratio of short to long fragments and a fragment size distribution from the fragmentation profile in the sample as correlated to a ratio from a sample from a healthy subject;
calculating a divergence score based on the ratio of short to long fragments;
determining a set of model weights based on a fragment size distribution;
training a machine learning model using a set of features extracted from fragmentation profiles of multiple subjects;
determining, by the machine learning model, a monitoring score for the sample based on the fragmentation score, the divergence score, and the model weights, the monitoring score being indicative of a level of a tumor-derived in cfDNA of the sample, thereby indicating a likelihood of cancer progression in the subject; and
determining at least one of overall survival, progression free survival, or time to progression based on the monitoring score.

23. A system for monitoring cancer in a subject, the system comprising:
a memory; and
one or more processors coupled to the memory, the one or more processors configured to perform operations that cause the computer system to:
determine a cell-free DNA (cfDNA) fragmentation profile of a sample from the subject;
calculate a fragmentation score based on the cfDNA fragmentation profile, the score being indicative of a likelihood of presence of cancer in the subject;
determine a ratio of short to long fragments and a fragment size distribution from the fragmentation profile in the sample as correlated to a ratio from a sample from a healthy subject;
calculate a divergence score based on the ratio of short to long fragments;
determine a set of model weights based on a fragment size distribution;
train a machine learning model using a set of features extracted from fragmentation profiles of multiple subjects; and
determine, by the machine learning model, a monitoring score for the sample based on the fragmentation score, the divergence score, and the model weights, the monitoring score being indicative of a level of a tumor-derived in cfDNA of the sample, wherein the level of tumor-derived in the cfDNA is indicative of a likelihood of cancer progression in the subject.
